(19) United States
(12) Patent Application Publication
(10) Pub. No.: US 2023/0256049 A1
(43) Pub. Date: Aug. 17, 2023

(54) MATERIALS AND METHODS FOR THE PREVENTION AND TREATMENT OF VIRAL RESPIRATORY DISEASES

(71) Applicant: OAK CREST INSTITUTE OF SCIENCE, Monrovia, CA (US)

(72) Inventors: Marc M. Baum, Altadena, CA (US); Manjula Gunawardana, Claremont, CA (US); John A. Moss, Sierra Madre, CA (US)

(21) Appl. No.: 18/017,582

(22) PCT Filed: Jul. 23, 2021
(86) PCT No.: PCT/US2021/043050

§ 371 Date: Jan. 23, 2023

Related U.S. Application Data
(60) Provisional application No. 63/180,405, filed on Apr. 27, 2021.
(60) Provisional application No. 63/056,301, filed on Jul. 24, 2020.

(51) Int. Cl. A61K 38/10; A61K 45/06; A61P 31/14

(52) U.S. Cl. CPC A61K 38/10; A61K 45/06; A61P 31/14

(57) ABSTRACT

This disclosure is related to the use of a peptide with antimicrobial properties. The peptide is administered topically via nasal or pulmonary delivery to prevent or treat respiratory diseases caused by viruses. A variety of formulations and uses are described as well as methods of manufacture thereof. The formulations are safe and useful in patients—both humans and animals—for the delivery of appropriate bioactive substance(s) to the respiratory system.

### CROSS-REFERENCE TO RELATED APPLICATIONS

This application claims priority to U.S. Patent Application No. 63/056,301, filed Jul. 24, 2020, and U.S. Patent Application No. 63/180,405, filed Apr. 27, 2021, the disclosures of which are hereby incorporated by reference in their entirety.

### FIELD OF INVENTION

This disclosure generally relates to the delivery of peptides to treat or prevent diseases and disorders.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “55883_Seqlisting.txt”, which was created on Jul. 23, 2021 and is 27,186 bytes in size. The subject matter of the Sequence Listing is incorporated herein in its entirety by reference.

### BACKGROUND

Respiratory viruses are responsible for a range of diseases including the common cold, certain forms of bronchitis and pneumonia, and influenza (“the flu”). The severity of symptoms varies significantly among infected individuals, ranging from mild symptoms to death. In addition to the loss of life, these diseases place a heavy economic burden on countries. In the U.S., the Centers for Disease Control and Prevention (CDC) estimated that the 2018-19 flu season led to 35.5 million people becoming ill, including 490,600 hospitalizations and 34,200 deaths (www.cdc.gov/flu/about/burden). The associated economic impact was large, including over $10 billion in hospitalization direct costs that do not take into account the loss in productivity due to morbidity.

The rapid spread of severe acute respiratory syndrome coronavirus 2 (SARS-CoV-2) and the associated coronavirus disease 2019 (COVID-19) has precipitated a global pandemic heavily challenging our social behavior, economy, and healthcare infrastructure. The number of confirmed COVID-19 cases and deaths are rising rapidly in different parts of the world, with tens of millions of individuals infected and hundreds of thousands of global deaths reported. Worldwide, recovery from this active, devastating outbreak cannot begin until safe and effective medicines for treatment and prevention are available. Currently, there are no approved drugs, immune therapeutics, or vaccines against SARS-CoV-2. Several vaccine candidates have been granted emergency use authorization by the FDA, others are being evaluated in clinical trials, while over 100 are under development (1). The global R&D effort is unprecedented in terms of scale and speed, but a number of potential complications remain that could delay the development of a safe and effective vaccine against SARS-CoV-2 (2), especially emerging new strains. These include toxicity from unexpected antibody-dependent enhancement (ADE) and T_{H}2 immunopathology (3, 4). Alternative therapies for COVID-19 prevention and treatment are needed urgently.

### SUMMARY

Provided herein are methods of treating or preventing a viral respiratory disease, the method comprising administering to a subject in need thereof a peptide comprising the amino acid sequence of Peptide 346-001 or variant thereof comprising one, two, or three amino acid substitutions, wherein the administration is via intranasal or pulmonary routes of administration.

Also provided are methods of treating or preventing a viral respiratory disease, the method comprising administering to a subject in need thereof a peptide comprising X₁-X₂-X₃-X₄-X₅-X₆-X₇-X₈-X₉-X₁₀-X₁₁-X₁₂-X₁₃-X₁₄-X₁₅-X₁₆-X₁₇-X₁₈ (Formula 1), wherein X₁ is S, K, D, A, N, M, T, or V; X₂ is W, I, Y, F, P, L, V, H, M, A, T, or S; X₃ is L, W, I, M, V, F, M, A, or T; X₄ is R, C, K, Q, H, P, or C; X₅ is D, R, I, E, N, or Y; X₆ is I, W, V, L, or F; X₇ is W, V, Y, F, P, L, V, H, or D; X₈ is D, S, E, N, Y, or S; X₉ is W, L, Y, F, P, L, V, or H; X₁₀ is I, V, L, or F; X₁₁ is C, S, A, P, M, H, or T; X₁₂ is E, D, S, Q, Y, or T; X₁₃ is V, F, I, L, A, F, or M; X₁₄ is L, V, I, M, or F; X₁₅ is S, D, A, N, T, Y, or P; X₁₆ is D, E, N, Y, or S; X₁₇ is F, W, Y, L, P, or H; and X₁₈ is K, E, R, H, R, P, or Q, wherein the administration is via intranasal or pulmonary routes of administration.

### BRIEF DESCRIPTION OF THE DRAWINGS

FIG. 1 shows examples of peptide conjugates based on Peptide 346-001 backbone. “Conj,” denotes conjugate.

FIG. 2 shows examples of general peptide N-conjugates via a lysine linker based on Peptide 346-001 backbone.

FIG. 3 shows examples of general peptide 0-conjugates via a serine linker based on Peptide 346-001 backbone.

FIG. 4 shows examples of general peptide S-conjugates via a cysteine linker based on Peptide 346-001 backbone.

FIG. 5A shows a sigmoidal Dose-Response (Variable Slope) Curve. This figure (means+SD, in triplicate) provides the dose-response curve for the experiment described in Example 3. Based on the best-fit, the following EC₅₀ values were calculated: (A) E SARS-CoV-2 target, 0.76 μM.

FIG. 5B shows a sigmoidal Dose-Response (Variable Slope) Curve. This figure (means+SD, in triplicate) provides the dose-response curve for the experiment described in Example 3. Based on the best-fit, the following EC₅₀ values were calculated: (B) orf8 SARS-CoV-2 target, 0.80 μM.

FIG. 5C shows a Dose-Response Relationship. This figure (means±SD, in triplicate) provides the dose-response relationship for the experiment described in Example 3 in terms of SARS-CoV-2 copy numbers measured. Black, E SARS-CoV-2 target; grey, orf8 SARS-CoV-2 target; vertical broken line represents the calculated EC₅₀ value (0.8 μM).

FIGS. 6A and 6B show a Sigmoidal Dose-Response (Variable Slope) Curve for Peptide 346-001. These figures (medians, in quadruplicate) provide the dose-response curve for the experiment described in Example 4. Based on the best-fit, the following EC₅₀ values were calculated: (A) E SARS-CoV-2 target, 20 μM; (B) orf8 SARS-CoV-2 target, 13 μM.

FIGS. 7A and 7B show a Sigmoidal Dose-Response (Variable Slope) Curve for Peptide 346-002. These figures (medians, in quadruplicate) provide the dose-response curve for the experiment described in Example 4. Based on the best-fit, the following EC₅₀ values were calculated: (A) E SARS-CoV-2 target, 14 μM; (B) orf8 SARS-CoV-2 target, 10 μM.

FIGS. 8A and 8B show a Sigmoidal Dose-Response (Variable Slope) Curve for Peptide 346-003. These figures (medians, in quadruplicate) provide the dose-response curve for the experiment described in Example 4. Based on the best-fit, the following EC₅₀ values were calculated: (A) E SARS-CoV-2 target, 40 μM; (B) orf8 SARS-CoV-2 target, 30 μM.

FIGS. 9A and 9B show a Sigmoidal Dose-Response (Variable Slope) Curve for Peptide 346-004. These figures (medians, in quadruplicate) provide the dose-response curve for the experiment described in Example 4. Based on the best-fit, the following EC₅₀ values were calculated: (A) E SARS-CoV-2 target, 16 μM; (B) orf8 SARS-CoV-2 target, 7.1 μM.

FIGS. 10A and 10B show a Sigmoidal Dose-Response (Variable Slope) Curve for Peptide 346-007. These figures (medians, in quadruplicate) provide the dose-response curve for the experiment described in Example 4. Based on the best-fit, the following EC₅₀ values were calculated: (A) E SARS-CoV-2 target, 17 μM; (B) orf8 SARS-CoV-2 target, 18 μM.

FIGS. 11A and 11B show a Sigmoidal Dose-Response (Variable Slope) Curve for Peptide 346-001. These figures (means±SD, in quadruplicate) provide the dose-response curve for the experiment described in Example 5. Based on the best-fit, the following EC₅₀ values were calculated: (A) E SARS-CoV-2 target, 16 μM; (B) orf8 SARS-CoV-2 target, 14 μM.

FIGS. 12A and 12B show a Sigmoidal Dose-Response (Variable Slope) Curve for Peptide 346-002. These figures (means±SD, in quadruplicate) provide the dose-response curve for the experiment described in Example 5. Based on the best-fit, the following EC₅₀ values were calculated: (A) E SARS-CoV-2 target, 14 μM; (B) orf8 SARS-CoV-2 target, 13 μM.

FIGS. 13A and 13B show a Sigmoidal Dose-Response (Variable Slope) Curve for Peptide 346-004. These figures (means±SD, in quadruplicate) provide the dose-response curve for the experiment described in Example 5. Based on the best-fit, the following EC₅₀ values were calculated: (A) E SARS-CoV-2 target, 18 μM; (B) orf8 SARS-CoV-2 target, 13 μM.

FIGS. 14A and 14B show a Sigmoidal Dose-Response (Variable Slope) Curve for Peptide 346-007. These figures (means±SD, in quadruplicate) provide the dose-response curve for the experiment described in Example 5. Based on the best-fit, the following EC₅₀ values were calculated: (A) E SARS-CoV-2 target, 8.6 μM; (B) orf8 SARS-CoV-2 target, 7.7 μM.

FIG. 15 shows a Dose-Response Relationship for Peptide 346-001 in Human Nasal Epithelial Culture. This figure (medians, in quadruplicate) shows the reduction in SARS-CoV-2 viral titer as a function of peptide concentration for the experiment described in Example 6.

FIG. 16 shows a Dose-Response Relationship for Peptide 346-002 in Human Nasal Epithelial Culture. This figure (medians, in quadruplicate) shows the reduction in SARS-CoV-2 viral titer as a function of peptide concentration for the experiment described in Example 6.

FIG. 17 shows a Dose-Response Relationship for Peptide 346-004 in Human Nasal Epithelial Culture. This figure (medians, in quadruplicate) shows the reduction in SARS-CoV-2 viral titer as a function of peptide concentration for the experiment described in Example 6.

FIG. 18 shows a Dose-Response Relationship for Peptide 346-007 in Human Nasal Epithelial Culture. This figure (medians, in quadruplicate) shows the reduction in SARS-CoV-2 viral titer as a function of peptide concentration for the experiment described in Example 6.

FIGS. 19A, 19B, 19C, and 19D show in vitro HIV-1 inhibition of antiviral peptides derived from Peptide 346-001. FIG. 19A shows data for Peptides 346-001, 346-040, 346-041, 346-043, and 346-044; FIG. 19B shows data for Peptides 346-001, 346-045, 346-046, 346-047, and 346-048; FIG. 19C shows data for Peptides 346-001, 346-049, 346-050, 346-052, and 346-053; and FIG. 19D shows data for Peptides 346-001, 346-054, 346-055, 346-056, and 346-057.

### DESCRIPTION OF THE INVENTION

The disclosure provides devices, systems and methods for treating, preventing, reducing the likelihood of having, reducing the severity of and/or slowing the progression of a condition in a subject. In various aspects, the disclosure provides materials and methods designed for the prevention and treatment of viral respiratory disease. Aspects of the materials and methods include, but are not limited to:
A peptide, or peptide prodrug, or peptide conjugate, comprising 5-50 amino acids with antimicrobial properties;
Optionally, one or more other active pharmaceutical ingredients (APIs; also referred to herein as “complementary agents”) used in combination with the above peptide; and
A drug delivery system that results in a pharmacologically desirable amount of the peptide (and, optionally API(s)) in the target anatomic compartment within the respiratory system.

Thus, in various aspects, the disclosure provides a method of treating or preventing a viral respiratory disease. In particular, the disclosure provides a method of treating or preventing a disease caused by a virus. Exemplary non-limiting virus families include Coronaviridae and Orthomyxoviridae. The method comprises administering to a subject in need thereof a peptide comprising (or consisting essentially of or consisting of) the sequence of Peptide 346-001 set forth in TABLE 1 below or variant thereof comprising one, two, or three amino acid substitutions. In various aspects, the peptide comprises the amino acid sequence of Formula 1:

X₁-X₂-X₃-X₄-X₅-X₆-X₇-X₈-X₉-X₁₀-X₁₁-X₁₂-X₁₃-X₁₄-
X₁₅-X₁₆-X₁₇-X₁₈ (Formula 1 (SEQ ID NO: 72),

wherein:
X₁ is S, K, D, A, N, M, T, or V; X₂ is W, I, Y, F, P, L, V, H, M, A, T, or S; X₃ is L, W, I, M, V, F, M, A, or T; X₄ is R, C, K, Q, H, P, or C; X₅ is D, R, I, E, N, or Y; X₆ is I, W, V, L, or F; X₇ is W, V, Y, F, P, L, V, H, or D; X₉ is D, S, E, N, Y, or S; X₉ is W, L, Y, F, P, L, V, or H; X₁₀ is I, V, L, or F; X₁₁ is C, S, A, P, M, H, or T; X₁₂ is E, D, S, Q, Y, or T; X₁₃ is V, F, I, L, A, F, or M; X₁₄ is L, V, I, M, or F; X₁₅ is S, D, A, N, T, Y, or P; X₁₆ is D, E, N, Y, or S; X₁₇ is F, W, Y, L, P, or H; and X₁₉ is K, E, R, H, R, P, or Q. The administration can be achieved by intranasal or pulmonary routes of administration. Optionally, the method comprises further administering one or more complementary agents (i.e., other APIs) suitable for, e.g., the treatment of a viral respiratory disease or related illness or symptom.

The prevention and treatment of respiratory viral infections with broad therapies is not always successful. For example, the treatment of SARS-CoV-2 with a chloroquine and hydroxychloroquine failed and led to increased patient morbidity risk, and remdesivir provided only a small beneficial impact in a double-blind, randomized, placebo-controlled trial in adults hospitalized with COVID-19 with evidence of lower respiratory tract involvement (24). Provided herein is the first disclosed use of the peptide disclosed herein to treat or prevent respiratory viruses.

Viral respiratory diseases are generally illnesses caused by viruses having similar traits and which affect the upper respiratory tract. Viral respiratory diseases include, e.g., infectious diseases (e.g., a coronavirus (CoV) infection (e.g., SARS-CoV-2 infection), an influenza infection, a parainfluenza infection, a respiratory syncytial virus (RSV) infection, a respiratory adenovirus infection, and the like), and analogous conditions in non-human mammals. The viral respiratory disease may be, for example, COVID-19, severe acute respiratory syndrome (SARS), or Middle East respiratory syndrome (MERS). Symptoms of the viral respiratory disease may include, e.g., fever, headache, body aches, dry cough, hypoxia (oxygen deficiency), and/or pneumonia. In some cases, the viral respiratory disease is COVID-19 (i.e., SARS-CoV-2 infection).

It will be appreciated that “treating” a disease or disorder does not require 100% abolition of the disease or disorder (i.e., complete reversal of the disease). Any degree of “treatment” is contemplated by the disclosure, including lessening of one or more symptoms of the disease, reduction in viral load, improvement in quality of life, and the like. Similarly, it will be appreciated that “preventing” a disease, in the context of the disclosure, does not require 100% inhibition of appearance of the disease. Any degree of reduction in the initial appearance of symptoms, inhibition of viral infection, prolonging relapse, inhibiting an initial surge of viral load, and the like, are contemplated.

In the context of the disclosure, the subject is a mammal, which refers to any member of the class Mammalia, including, without limitation, humans and nonhuman primates such as chimpanzees and other apes and monkey species; farm animals such as cattle, sheep, pigs, goats and horses; domesticated mammals, such as dogs and cats; laboratory animals including rodents such as mice, rats and guinea pigs, and the like. In some cases, the subject is a human. In some cases, the subject is a non-human mammal. The term does not denote a particular age or sex. Thus, adults (i.e., human subjects aged 18 years or more), children (i.e., human subjects aged one to eighteen years) and newborns (i.e., human subjects aged one year or less), whether male or female, are intended to be included within the scope of “subject.”

### Antiviral Peptides

One preferred set of embodiments of the disclosure involves the delivery of peptides with antiviral properties. Optionally, the peptides have a length of five to fifty amino acid residues (e.g., five to eight amino acid residues; or eight to twelve amino acid residues; or twelve to twenty amino acid residues; or twenty to thirty five amino acid residues; or thirty five to fifty amino acid residues). In one exemplary aspect of the disclosure, the antimicrobial peptide comprises (or consists of, or consists essentially of) the amino acid sequence: SWLRDIWDWICEVLSDFK (SEQ ID NO: 1) (Peptide 346-001, TABLE 1). The amino acid sequence corresponds to a virucidal amphipathic α-helical peptide derived from the hepatitis C virus (HCV) NS5A membrane anchor domain, and is referenced herein as “C5A” (5, 6), or Peptide 346-001 (TABLE 1). The disclosure also contemplates use of a peptide derived from C5A, which comprises a sequence substantially identical to the sequence of Peptide 346-001. In this regard, the disclosure contemplates, e.g., a variant of Peptide 346-001 comprising, e.g., one, two, or three amino acid substitutions (such as conservative substitutions). Conservative substitutions, deletions and additions may be made at non-critical residue positions within the selected peptide without substantially adversely affecting its biological activity. Similar modifications can be made at the receptor binding site(s) to augment binding efficiency and specificity. In addition, changes may be made to select residues to increase peptide stability (e.g., replacing a cysteine residue with serine). The peptide can be optionally flanked and/or modified at one or both of the N- and C-termini, as desired.

Optionally, the peptide comprises the amino acid sequence of Peptide 346-001 comprising one, two, or three substitutions selected from the following: the S at position 1 (X₁) is substituted with K, D, A, N, M, T, or V; the W at position 2 (X₂) is substituted with I, Y, F, P, L, V, H, M, A, T, or S; the L at position 3 (X₃) is substituted with W, I, M, V, F, M, A, or T; the R at position 4 (X₄) is substituted with C, K, Q, H, P, or C; the D at position 5 (X₅) is substituted with R, I, E, N, or Y; the I at position 6 (X₅) is substituted with W, V, L, or F; the W at position 7 (X₇) is substituted with V, Y, F, P, L, V, H, or D; the D at position 8 (X₈) is substituted with S, E, N, Y, or S; the W at position 9 (X₉) is substituted with L, Y, F, P, L, V, or H; the I at position 10 (X₁₀) is substituted with V, L, or F; the C at position 11 (X₁₁) is substituted with S, A, P, M, H, or T; the E at position 12 (X₁₂) is substituted with D, S, Q, Y, or T; the V at position 13 (X₁₃) is substituted with F, I, L, A, F, or M; the L at position 14 (X₁₄) is substituted with V, I, M, or F; the S at position 15 (X₁₅) is substituted with D, A, N, T, Y, or P; the D at position 16 (X₁₆) is substituted with E, N, Y, or S; the F at position 17 (X₁₇) is substituted with W, Y, L, P, or H; or the K at position 18 (X₁₈) is substituted with E, R, H, R, P, or Q. In various aspects, the peptide comprises the amino acid sequence of Peptide 346-001 comprising a substitution at position 11 such that the cysteine is substituted with another amino acid (i.e., X₁₁ is not C). Optionally, the peptide comprises the amino acid sequence of Peptide 346-001 wherein the C at position 11 (X₁₁) is substituted with S, A, P, M, H, or T. Also optionally, the peptide includes D forms of any of the amino acids described herein (e.g., all or a subset of the amino acids may be D-amino acids).

In various aspects, the peptide has a sequence of Formula 1:

X₁-X₂-X₃-X₄-X₅-X₆-X₇-X₈-X₉-X₁₀-X₁₁-X₁₂-X₁₃-X₁₄-
X₁₅-X₁₆-X₁₇-X₁₈ (Formula 1 (SEQ ID NO: 72)),

wherein:
X₁ is S, K, D, A, N, M, T, or V; X₂ is W, I, Y, F, P, L, V, H, M, A, T, or S; X₃ is L, W, I, M, V, F, M, A, or T; X₄ is R, C, K, Q, H, P, or C; X₅ is D, R, I, E, N, or Y; X₆ is I, W, V, L, or F; X₇ is W, V, Y, F, P, L, V, H, or D; X₉ is D, S, E, N, Y, or S; X₉ is W, L, Y, F, P, L, V, or H; X₁₀ is I, V, L, or F; X₁₁ is C, S, A, P, M, H, or T; X₁₂ is E, D, S, Q, Y, or T; X₁₃ is V, F, I, L, A, F, or M; X₁₄ is L, V, I, M, or F; X₁₅ is S, D, A, N, T, Y, or P; X₁₆ is D, E, N, Y, or S; X₁₇ is F, W, Y, L, P, or H; and X₁₈ is K, E, R, H, R, P, or Q.

In various aspects, the peptide comprises a sequence set forth in Table 1 or in the sequence listing provided herewith, which is hereby incorporated by reference. The sequence may include L or D forms of the amino acids, or any combination thereof. The disclosure also contemplates peptides wherein the sequence set forth in Table 1 (e.g., Peptide 346-001) is reversed (i.e., a retropeptide, such as a peptide comprising the reversed sequence of Peptide 346-001 such that the N-terminus listed in Table 1 is the C terminus of the retropeptide).

Peptide sequences derived from Peptide 346-001, and modifications thereof, may possess different, non-obvious pharmacologic properties relative to the parent sequence. These may consist of higher or lower in vitro and/or in vivo efficacy against one or more viruses that cause respiratory diseases. Other, exemplary properties that may be affected by modifying the peptide sequence include: in vitro stability; in vivo stability; in vivo half-life; protein binding; and cellular penetration. Non-limiting examples of peptide sequences against respiratory viruses are shown in TABLE 1. Non-obvious differences in the in vitro anti-SARS-CoV-2 properties of some of these peptide are disclosed under EXAMPLES 4-6.

In non-limiting aspects of the disclosure, the peptide contains overall features that impact its efficacy against viruses. One exemplary, non-limiting feature is associated with the peptide's α-helical structure. Without wishing to be bound by any particular theory, the α-helical structure can allow the peptide to interact with the viral membrane and disrupt its integrity, thereby releasing viral components such as capsids and exposing the viral genetic material to exonuclease degradation. The charge distribution along the peptide's α-helix, determined by the nature of the amino acid side-groups in the sequence, is an exemplary feature that may determine how the peptide associates, binds, and/or disrupts the viral particle. Another exemplary, non-limiting feature concerns the peptide's amphipathic nature, defined commonly in the art as possessing both hydrophilic and lipophilic properties. Without wishing to be bound by any particular theory, the peptide's amphipathicity can disrupt the ligand-host receptor interaction and viral escape from endosomes. The overall peptide hydrophobicity is another exemplary feature that can impact its antiviral properties by determining how it recognizes host-cellular components of virus membranes, possibly by their lipid composition. Another exemplary, non-limiting feature is associated with specific peptide-membrane protein interaction, such that retropeptides, peptides with scrambled hydrophobic or hydrophilic amino acids, or peptides comprised containing D-amino acids, but all based on the sequence of the parent, active peptide (e.g., Peptide 346-001), display antiviral properties. In summary, there are a number of features that can, in certain embodiments, affect the antiviral peptide's interaction with the viral particle, specifically the viral membrane or another feature on the virus surface, causing disruption and leading to virus inactivation. In some cases, destabilization of physical linkages between the mature conical capsid core and the viral membrane, i.e., the core-membrane linkage, can occur. In other cases, the shedding of the viral surface protein(s) responsible for entry into cells can be altered or shed.

As used herein, the term “peptide” refers to a series of amino acids connected one to the other by peptide bonds between the α-amino and α-carboxy groups of adjacent amino acids. Peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described. Although the peptides described herein, in various aspects, are substantially free of other naturally occurring proteins and fragments thereof, in some embodiments the peptides can be synthetically conjugated to native fragments or particles.

In the various embodiments the peptides are optionally polymerized, each to itself, to form larger homopolymers, or with different peptides to form heteropolymers. In some instances, peptides will be combined in a composition as an admixture and will not be linked. The peptide can also be conjugated to lipid-containing molecules or to different peptides.

In an aspect of the disclosure, the peptide is chemically conjugated with a moiety that provides a functional or practical advantage. Non-limiting examples of such linked species, known in the art, include: polyethylene glycol, or other polymers to increase the in vivo residence time or half-life of the peptide; a small, biocompatible linker group that is amenable to high yielding bioconjugation reactions, so-called “click chemistry” linkers well known in the art (7-10) and fully incorporated herein; a biotin linker on the peptide that binds to streptavidin on another moiety, or other similar affinity-based linker systems known in the art, to facilitate in vivo detection or imaging of the peptide.

Linkages for homo- or hetero-polymers or for coupling to carriers can be provided in a variety of ways known in the art. For example, cysteine residues can be added at both the amino- and carboxy-termini, where the peptides are covalently bonded via controlled oxidation of the cysteine residues. Also useful are a large number of heterobifunctional agents which generate a disulfide link at one functional group end and a peptide link at the other, including N-succidimidyl-3-(2-pyridyl-dithio) propionate (SPDP). This reagent creates a disulfide linkage between itself and a cysteine residue in one protein and an amide linkage through the amino on a lysine or other free amino group in the other.

In various aspects, the pharmacology of the peptide is enhanced by synthesizing a suitable prodrug; methods of prodrug synthesis are known in the art (11-13). Prodrugs of the peptide can involve the C-terminal carboxy group, the tyrosine phenol group in tyrosyl peptides, and the N-terminal amino group. The peptide prodrug can have improved pharmacological properties when compared to the parent drug, such as improved bioavailability to the target compartment.

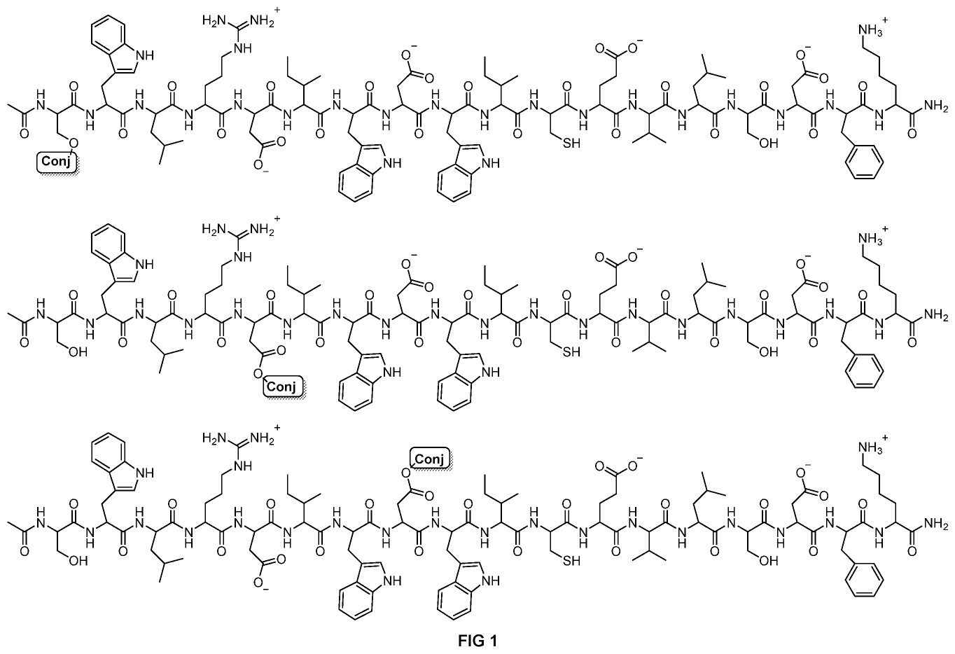

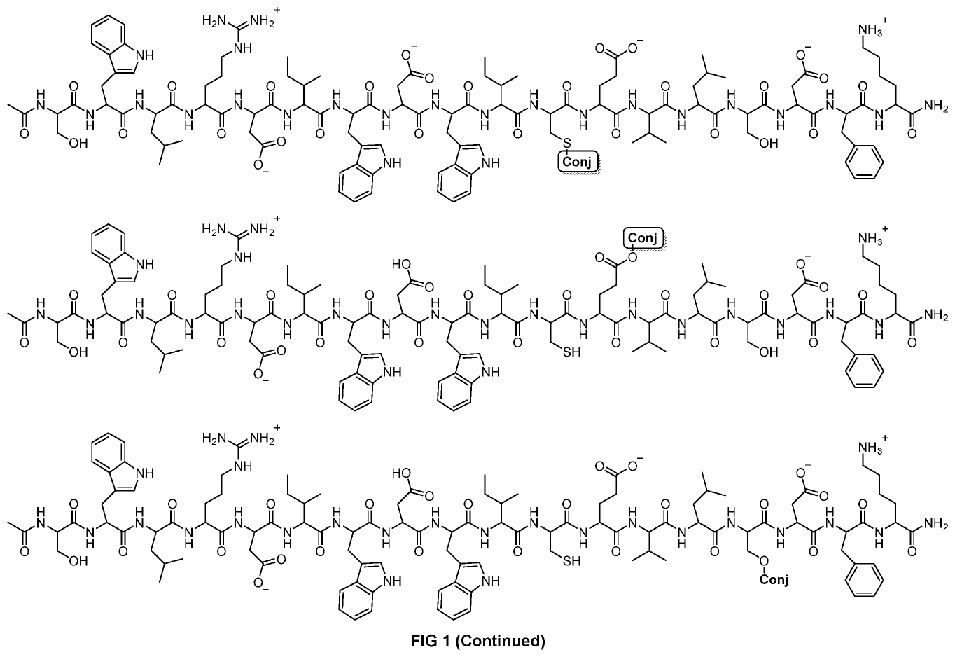

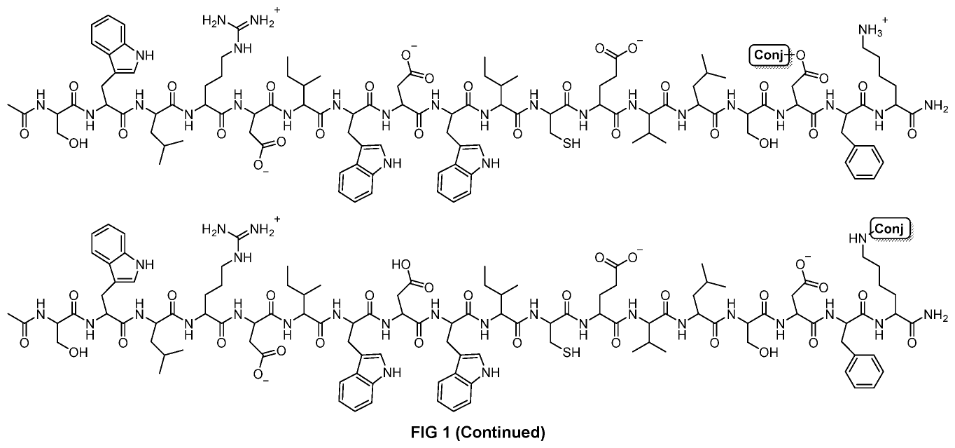

In various aspects of the disclosure, the pharmacology of the peptide is enhanced by synthesizing a suitable conjugate with a lipophilic moiety, or an acceptable salt thereof, as shown schematically in TABLE 2 using Peptide 346-001 as an exemplary backbone. It is understood that this approach also applies to the other exemplary, non-limiting peptides shown in TABLE 1. In accordance with TABLE 2, peptide conjugation can occur in a variety of non-limiting strategies. Conjugation can be beneficial at the N-terminus, at the C-terminus, or at both termini. Conjugation can also be beneficial at the reactive side chains of the peptide, as shown in FIG. 1 using Peptide 346-001 as an exemplary backbone. Any combination of these conjugation strategies can be used to enhance the properties of the parent peptide backbone. The conjugates are prepared using organic synthesis strategies, methods, and processes, many of which are known in the art.

In various aspects, conjugation is achieved directly to the peptide. In various aspects, conjugation is achieved via one or more linker groups. Optionally, the linker, or spacer, consists of a single amino acid. Alternatively, the linker optionally comprises two or more amino acids, such as GSG, multiples thereof (GSG)ₙ, or GSGSGC (SEQ ID NO: 73), known in the art. As described herein, linkers can comprise peptides, polyether compounds (i.e., PEG), and/or combinations thereof. In applications where it is beneficial to have longer distance between the conjugate and the antiviral peptide, polyethylene glycol (PEG) chains of varying lengths. PEG is a typically biologically inert chemical that confers greater water solubility to peptides with which it is incorporated as constituent chemical group. PEG is non-toxic and non-immunogenic, hydrophilic, and highly flexible. In additional embodiments, the linker comprises one or more polyethylene glycol oligomer moieties having a formula of —(OCH₂CH₂)ₘ—, wherein m is an integer 1 to 24. In one exemplary embodiment, m is 24-1,000. In another exemplary embodiment, m is 1,000-5,000.

In accordance with this aspect of the present disclosure, linkers may be an amino acid, such as, but not limited to lysine, serine, or cysteine.

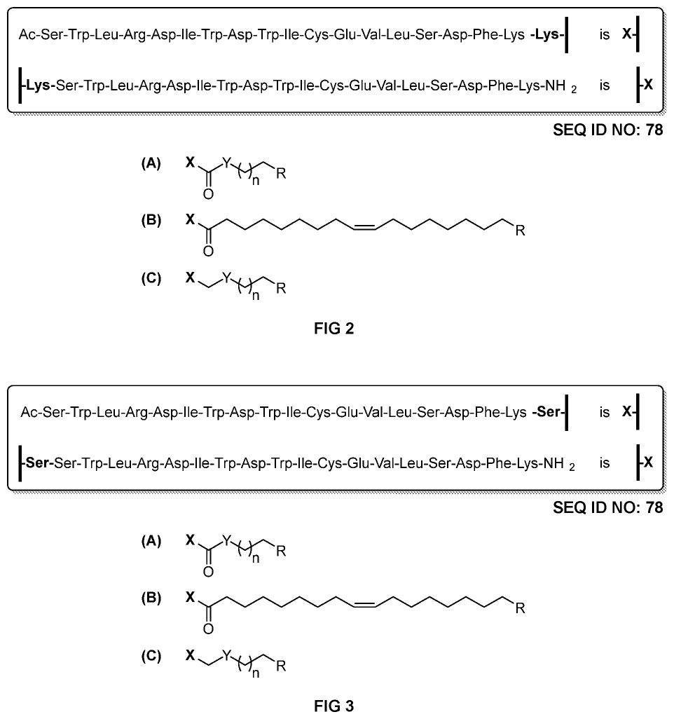

When lysine is used as a linker, either as a single amino acid or as part of a larger linker comprising two or more amino acids, synthetic spacers (e.g., PEG) or a combination thereof, the amino side-chain can be used for conjugation as shown in FIG. 2 using the Peptide 346-001 backbone as a non-limiting example. Suitable conjugates can be carbonyl compounds, as depicted in FIGS. 2 (A) and (B). In such non-limiting embodiments, Y may be O, S, NR₁ (where R₁ is H, alkyl, alkyl-aryl (e.g., benzyl), or aryl), P(O)R₂ (where R₂ is alkyl, alkyl-aryl (e.g., benzyl), aryl, OH, O-alkyl, O-alkyl-aryl, O-aryl, NR₁-alkyl, NR₁-alkyl-aryl, or NR₁-aryl), or CR₂R₃ (where R₂ and or R₃ are H, alkyl, alkyl-aryl (e.g., benzyl), aryl, O-alkyl, O-alkyl-aryl, O-aryl, or NR₁-alkyl, NR₁-alkyl-aryl, or NR₁-aryl). In other words, as described herein, exemplary, non-limiting conjugates as shown in FIG. 2 can be classified as amides, carbamates, ureas, thiocarbamates, phosphoramidates, and phosphonates. The alkyl chain length, n, is 1-50 and R is alkyl, aryl, or derivatives thereof, such as PEG. In one non-limiting example, palmitic acid is conjugated with the lysine linker; i.e., Y═CH₂, n=13, R═CH₃. In non-limiting embodiments, the moiety conjugated to the peptide is unsaturated, such as oleic acid (R═CH₃) as shown in FIG. 2 (B) or linoleic acid, and comprises one or more double bonds. In other non-limiting examples, the lysine NH₂ side chain is conjugated via a methylene group, as shown in FIG. 2 (C), and Y can be O, S, NR₁, P(O)R₂, or CR₂R₃.

In non-limiting examples, the moiety conjugated to X via the O═C—Y— group as described above and in FIG. 2(A), or via the CH₂—Y— group as described above in FIG. 2(C) comprises one or more cholesterol groups, or derivatives thereof. In further non-limiting examples, the moiety comprises one or more tocopherol groups, or derivatives thereof. Tocopherols constitute a series of related benzopyranols (or methyl tocols) that occur in plant tissues and vegetable oils and are powerful lipid-soluble antioxidants. These compounds are produced by plants and other oxygenic photosynthetic organisms, such as algae and some cyanobacteria, and are essential components of the diet of animals, and collectively they are termed “vitamin E”. The antiviral peptide conjugate of the present disclosure includes tocopherol, a tocopherol derivative, or pharmaceutically acceptable salt thereof. Examples of tocopherol that may be used in the present aspect include, but are not limited to, α-tocopherol, δ-tocopherol, γ-tocopherol, and δ-tocopherol. Examples of useful tocopherol derivatives or pharmaceutically acceptable salts thereof include, but are not limited to, tocotrienols, tocopheroxyl radical, 8α-alkyldioxytocopherone, tocopherol quinone, tocopherol hydroquinone, D,L-tocopherol, D,1-tocopheryl acetate, α-tocopheryl acetate, α-tocopheryl nicotinate, and α-tocopheryl succinate. In further non-limiting examples, the moiety comprises one or more dihydrosphingosine groups, one or more sphingosine groups, or derivatives thereof. In further non-limiting examples, the moiety comprises one or more phospholipid groups, or derivatives thereof.

When serine is used as a linker, either as a single amino acid or as part of a larger linker comprising two or more amino acids, synthetic spacers (e.g., PEG) or a combination thereof, the hydroxy side-chain can be used for conjugation as shown in FIG. 3 using the Peptide 346-001 backbone as a non-limiting example. Suitable conjugates include carbonyl compounds, as depicted in FIGS. 3(A) and (B). In such non-limiting embodiments, Y may be O, S, NR₁ (where R₁ is H, alkyl, alkyl-aryl (e.g., benzyl), or aryl), P(O)R₂ (where R₂ is alkyl, alkyl-aryl (e.g., benzyl), aryl, OH, O-alkyl, O-alkyl-aryl, O-aryl, NR₁-alkyl, NR₁-alkyl-aryl, or NR₁-aryl), or CR₂R₃ (where R₂ and or R₃ are H, alkyl, alkyl-aryl (e.g., benzyl), aryl, O-alkyl, O-alkyl-aryl, O-aryl, or NR₁-alkyl, NR₁-alkyl-aryl, or NR₁-aryl). In other words, as described herein, exemplary, non-limiting conjugates as shown in FIG. 2 can be classified as esters, carbonates, carbamates, thiocarbonates, phosphoramidates, and phosphonates. The alkyl chain length, n, is 1-50 and R is alkyl, aryl, or derivatives thereof, such as PEG. In one non-limiting example, palmitic acid is conjugated with the serine linker; i.e., Y═CH₂, n=13, R═CH₃. In non-limiting embodiments, the moiety conjugated to the peptide is unsaturated, such as oleic acid (R═CH₃) as shown in FIG. 3 (B) or linoleic acid, and comprises one or more double bonds. In other non-limiting examples, the serine OH side chain is conjugated via a methylene group, as shown in FIG. 3 (C), and Y can be O, S, NR₁, P(O)R₂, or CR₂R₃.

In non-limiting examples the moiety conjugated to X via the O═C—Y— group as described above and in FIG. 3 (A), or via the CH₂—Y— group as described above in FIG. 3 (C) comprises one or more cholesterol groups, or derivatives thereof. In further non-limiting examples, the moiety comprises one or more tocopherol groups, or derivatives thereof. In further non-limiting examples, the moiety comprises one or more dihydrosphingosine groups, one or more sphingosine groups, or derivatives thereof. In further non-limiting examples, the moiety comprises one or more phospholipid groups, or derivatives thereof.

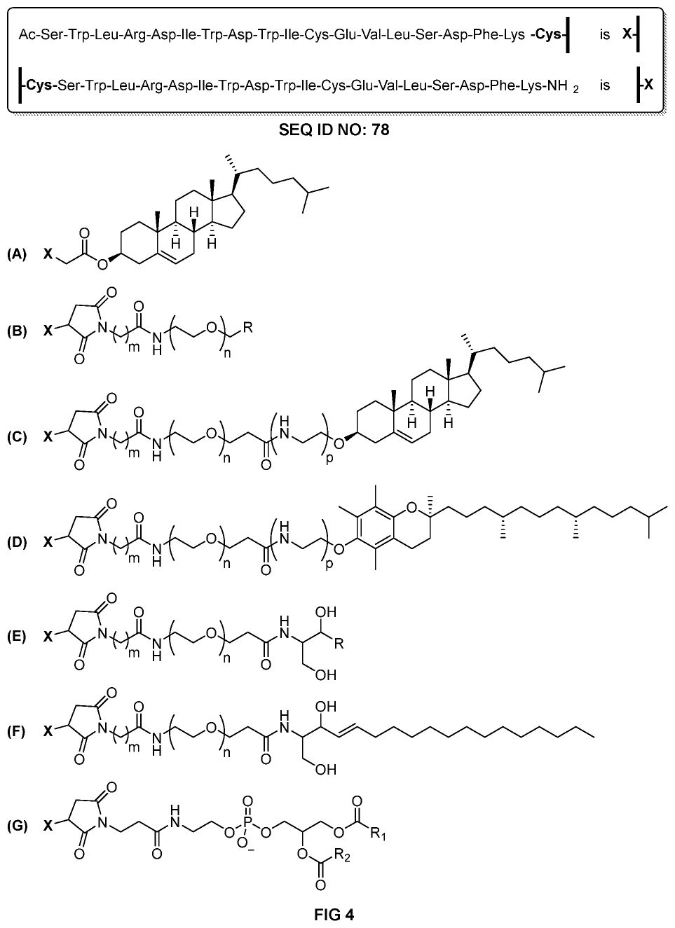

In general, thiol groups present in cysteine (or cysteine derivative) side-chains or terminal groups can be reacted with reagents possessing thiol-reactive functional groups using reaction schemes known in the art. Exemplary thiol-reactive functional groups include, without limitation, iodoacetamides, maleimides, and alkyl halides. Non-limiting examples of such conjugation schemes using a terminal cysteine linker are shown in FIG. 4. Cysteine can be used as a linker, either as a single amino acid or as part of a larger linker comprising two or more amino acids, synthetic spacers (e.g., PEG) or a combination thereof. FIG. 4 uses the SEQ. ID 346-001 backbone as a non-limiting example. Exemplary, non-limiting embodiments shown in FIG. 4 include: (A) derivatives of cholesterol, or pharmaceutically acceptable salts thereof; (B) maleimide derivatives of PEG, or pharmaceutically acceptable salts thereof, where m is 1-12, n is 1-12, and R is H, alkyl, or aryl; (C) maleimide derivatives of cholesterol, or pharmaceutically acceptable salts thereof, where m is 1-12, n is 1-12, and p is 1-12; (D) maleimide derivatives of tocopherol, or pharmaceutically acceptable salts thereof, where m is 1-12, n is 1-12, and p is 1-12; (E) maleimide derivatives of dihydrosphingosine, or pharmaceutically acceptable salts thereof, where m is 1-12, n is 1-12, and R is H, alkyl, or aryl; (F) maleimide derivatives of sphingosine, or pharmaceutically acceptable salts thereof, where m is 1-12, and n is 1-12; and (G) maleimide derivatives of phospholipids, or pharmaceutically acceptable salts thereof, where R is H, alkyl, or aryl. The peptides of the disclosure can be prepared in a wide variety of ways. In various aspects, the peptide is desirably small while maintaining substantially all of the virucidal activity of the large peptide. The peptides can be prepared synthetically or by recombinant DNA technology using, e.g., methods known in the art. The peptides can be synthesized in solution or on a solid support in accordance with conventional techniques. Various automatic synthesizers are commercially available and can be used in accordance with known protocols.

Compositions are also provided that comprise a peptide of the disclosure formulated with an additional peptide, a liposome, an adjuvant and/or a pharmaceutically acceptable carrier. Liposomes can also be used to increase the half-life of the peptide composition. Liposomes useful in the context of the present disclosure include emulsions, foams, micelles, insoluble monolayers, liquid crystals, phospholipid dispersions, lamellar layers and the like. In these preparations the peptide to be delivered is incorporated as part of a liposome, alone or in conjunction with a molecule that binds to a suitable receptor, or with other therapeutic or immunogenic compositions. Thus, liposomes filled with a desired peptide of the disclosure can be directed to the site of cells infected with the virus, where the liposomes then deliver the selected therapeutic/immunogenic peptide compositions. Liposomes for use in the contest of the disclosure may be formed from standard vesicle-forming lipids, which generally include neutral and negatively charged phospholipids and a sterol, such as cholesterol. The selection of lipids is generally guided by consideration of, e.g., liposome size and stability of the liposomes in the respiratory system. A variety of methods are available for preparing liposome, many of which are described in the art.

In another embodiment, the peptide is chemically or physically attached to polymeric micro- or nanoparticles, a number of which are known in the art. Nonlimiting examples include biodegradable, biocompatible microspheres and nanospheres, where nonlimiting examples of resorbable synthetic polymers include poly(lactic acids), poly(glycolic acids), poly(lactic-co-glycolic acids), poly(caprolactones) (PCLs), and mixtures thereof. In some embodiments, the peptide is dispersed inside the polymer particles using emulsion techniques well known in the art. In other embodiments, the N-terminus of the peptide is conjugated to the surface of the polymer particles via free carboxy groups. Alternatively, the peptide C-terminus is coupled to free carboxy groups on the surface of the polymer particles via a suitable linker group using synthetic chemistry approaches well-known in the art. The purposes of the polymer micro- or nanoparticles include, but are not limited to: monodispersity for efficient delivery to target regions of the respiratory system, size that allows for efficient pulmonary or nasal delivery (i.e., targeted delivery), and mucoadhesive properties to affect retention times of the agent.

### Complementary Agents

In addition to the peptide disclosed above, one or more complementary agents are included in certain embodiments of the disclosure. “Complementary” is intended to mean that the agents are supportive in achieving the desired pharmacological or biological effect (e.g., alleviating one or more symptoms of a viral respiratory disease, alleviating one or more side-effects of the disease, addressing a separate illness or disorder, or addressing an associated disruption in normal functioning of the subject).

In one embodiment, these complementary agents possess antimicrobial properties. Nonlimiting examples of complementary antimicrobial agents include:
Transition metal salts and complexes (e.g., zinc complexes, silver complexes, copper complexes);
Small-molecule antiviral agents and prodrugs thereof, active against influenza virus (e.g., oseltamivir phosphate, zanamivir, favipiravir, peramivir, and baloxavir marboxil);
Broad-based nucleoside inhibitor, and prodrugs thereof (e.g., acyclovir, ganciclovir, remdesivir, EIDD-1931/EIDD-2801, or ribavirin);
Antiretroviral agents used to treat HIV/AIDS, non-limiting examples of which include protease inhibitors;
Other antimicrobial agents and prodrugs thereof (e.g., niclosamide, chloroquine, hydroxychloroquine, carrageenan); and
Agents, and prodrugs thereof, that that modulate host responses to a viral infection, including, but not limited to didemnins (e.g., plitidepsin) and inhibitors of the rho-associated coiled-coil containing kinase 2 (ROCK2).

In one embodiment of the disclosure, the complementary agent comprises or consists of a protein with beneficial properties to prevent or treat the respiratory virus infection. In one non-limiting embodiment, the protein possesses broad antimicrobial properties. In one non-limiting example, the protein is natural lectin griffithsin (GRFT, UniProtKB/Swiss-Prot: P84801) or an engineered form of wild type GRFT, Q-GRFT, with improved oxidation resistance. In another embodiment, the complementary antiviral protein is a cytokine. In a non-limiting example, the cytokine is from the interferon family, such as interferon beta-1a. In another non-limiting example, the complementary agent comprises a so-called zinc finger antiviral agent, including zinc finger proteins, known in the art to enhance the efficacy of other peptides and proteins, including interferons as described by Nchioua et al. (14). In another embodiment, the complementary agent is an antiviral antibody. In non-limiting examples, the antibodies are polyclonal or monoclonal antibodies against spike, envelope, and/or nucleocapsid viral proteins.

In certain embodiments, complementary agents dampen the host's immune response to the viral infection. Such agents include steroids or derivatives thereof, such as, but not limited to, dexamethasone and fluticasone propionate. In another embodiment, such agents include non-steroidal anti-inflammatory drugs (NSAIDs) including, but not limited to aspirin, ibuprofen, celecoxib, diclofenac, and indomethacin.

In some cases, the complementary agent is an agent that affects immune and fibrotic processes. Non-limiting examples of agents that affect immune and fibrotic processes include inhibitors of rho-associated coiled-coil kinase 2 (ROCK2), for example, KD025 (Kadmon).

In some cases, the complementary agent is a sirtuin (SIRT1-7) inhibitor. In some cases, the sirtuin inhibitor is EV-100, EV-200, EV-300, or EV-400 (Evrys Bio). In some cases, administration of a sirtuin inhibitor restores a human host's cellular metabolism and immunity.

The complementary agents described herein can be administered alone (as part of a therapeutic regimen including administration of the peptide) or in combination with other complementary agents (and administration of the peptide). In some cases, the formulations described herein comprise more than one pharmaceutically active substance. In some cases, the formulations described herein comprise a combination of pharmaceutically active substances. In some cases, a combination of complementary agents is provided which includes chloroquine and azithromycin, hydroxychloroquine and azithromycin, lopinavir and ritonavir, KD025 and ribavirin, KD025 and remdesivir, EV-100 and ribavirin, or EV-100 and remdesivir. In certain embodiments, an antiviral peptide described herein is combined with one or more complementary agents disclosed above.

In certain aspects of the disclosure, a complementary agent is chemically linked with the peptide to generate peptide-drug conjugates. This strategy is an effective prodrug strategy for targeted delivery and improved pharmacological outcomes as described, for example, in the review by Wang et al. (15), incorporated herein by reference in its entirety and in particularly in regard to its discussion of conjugates.

### Respiratory Drug Delivery Systems

The active agent(s) described above (i.e., the antiviral peptide and complementary agents, when present) are administered to the respiratory system using any suitable delivery system. Suitable delivery systems include, but are not limited to, metered-dose aerosol systems, dry-powdered inhalation systems, and nasal inhalation systems. An illustrative nonlimiting summary of the various drug delivery embodiments is given below.

### Nasal Drug Delivery

Existing and emerging nasal drug delivery devices and concepts of aerosol generation for clinical have been reviewed, such as by Djupesland (16), incorporated by reference herein in its entirety. Nasal delivery of liquid and solid formulations of the agent(s) disclosed above (i.e., the antiviral peptide and/or complementary agent) can be achieved by the following non-limiting devices:
Pipettes for drop or vapor delivery, which may be breath powered or hand-actuated;
Vapor inhaler, such as commercial menthol inhalers for rhinitis;
Mechanical liquid spray pumps and squeeze bottles, which may be breath powered or hand-actuated;
Gas-driven spray systems and atomizers, including pressurized metered-dose inhalers (pMDIs);
Electrically powered nebulizers and atomizers;
Hand-actuated, single dose mechanical powder sprayers;
Breath-actuated solid inhalers; and
Single-dose mechanical solid insufflators.

### Pulmonary Drug Delivery

The mass median aerodynamic diameter and geometric standard deviation (GSD) are parameters that determine the site of particle deposition in the respiratory tract. Large particles or droplets deposit by impaction in the upper respiratory tree of the lung (oropharyngeal and tracheo-bronchial region), where air velocity is high and the air flow is turbulent. Particles in the size range of 0.5-5 μm deposit by sedimentation in the terminal bronchioles and alveolar regions. The larger the GSD, the more sites the aerosol will be deposited in the respiratory tract. In general, aerosols with GSD<2 are desirable and, ideally, aerosol particle size distributions should be as close as possible to monodispersity to increase deposition at the desired site of action and increase the efficacy of the treatment.

Pulmonary drug delivery strategies have been described and are well-known in the art (17-19). Dry powder inhalers are popular devices used to deliver drugs, especially proteins, to the lungs. Some of the commercially available dry powder inhalers include Spinhaler (Fisons Pharmaceuticals, Rochester, N.Y.) and Rotahaler (GSK, Research Triangle Park, N.C.). Several types of nebulizers are available, namely jet nebulizers, ultrasonic nebulizers, and vibrating mesh nebulizers. Jet nebulizers are driven by compressed air. Ultrasonic nebulizers use a piezoelectric transducer to create droplets from an open liquid reservoir. Vibrating mesh nebulizers use perforated membranes actuated by an annular piezo-element to vibrate in resonant bending mode. The holes in the membrane have a large cross-section size on the liquid supply side and a narrow cross-section size on the side from where the droplets emerge. Depending on the therapeutic application, the hole sizes and number of holes can be adjusted. Selection of a suitable device depends on parameters, such as nature of the drug and its formulation, the site of action, and pathophysiology of the lung. Aqueous suspensions and solutions are nebulized effectively. Aerosols based on mechanically generated vibration mesh technologies also have been used successfully to deliver proteins to lungs.

### Excipients and Manufacturing Considerations

### Excipients

Pharmaceutically acceptable excipients are known in the art and may include, e.g., viscosity modifiers, bulking agents, surface active agents, dispersants, disintegrants, osmotic agents, diluents, binders, anti-adherents, lubricants, glidants, pH modifiers, antioxidants and preservants, and other non-active ingredients of the formulation intended to facilitate handling and/or affect the release kinetics of the drug.

In some embodiments, the binders and/or disintegrants may include, but are in no way limited to, starches, gelatins, carboxymethylcellulose, croscarmellose sodium, methyl cellulose, ethyl cellulose, hydroxymethyl cellulose, hydroxyethyl cellulose, hydroxypropyl cellulose, hydroxypropylmethyl cellulose, hydroxypropylethyl cellulose, hydroxypropylmethyl cellulose, microcrystalline cellulose, polyvinylpyrrolidone, polyethylene glycol, sodium starch glycolate, lactose, sucrose, glucose, glycogen, propylene glycol, glycerol, sorbitol, polysorbates, and/or colloidal silicon dioxide. In certain embodiments, the anti-adherents or lubricants may include, but are in no way limited to, magnesium stearate, stearic acid, sodium stearyl fumarate, and/or sodium behenate. In some embodiments, the glidants may include, but are in no way limited to, fumed silica, talc, and/or magnesium carbonate. In some embodiments, the pH modifiers may include, but are in no way limited to, citric acid, lactic acid, and/or gluconic acid. In some embodiments, the antioxidants and preservants may include, but are in no way limited to ascorbic acid, butylated hydroxytoluene (BHT), butylated hydroxyanisole (BHA), cysteine, methionine, vitamin A, vitamin E, sodium benzoate, and/or parabens.

In some embodiments, excipients can stabilize biomolecules with respect to degradation or loss of biological activity using approaches known to those skilled in the art (20). Certain excipients stabilize biomolecules by creating a “water-like” environment in the dry state through hydrogen bonding interactions, e.g., sugars (21) and amino acids (22). Other excipients create a glassy matrix that provides hydrogen bonding and immobilized the biomolecules to prevent aggregation that leads to loss of biologic activity (e.g., trehalose, inulin). Still other excipients can stabilize the pH in the implant formulation (e.g., buffer salts). Finally, surfactants can reduce the concentration of the biomolecules at the air-water interface during drying processes of formulation, decreasing shear stress and insoluble aggregate formation, and allowing the previously described stabilization mechanisms to occur throughout the drying process.

### Manufacture of Dry Powder Formulations

Solid formulations for dry powder inhalers (DPIs) can be prepared by a variety of methods, many of which are well known in the art. These include, but are not limited to spray drying, spray-freeze drying, supercritical fluid technology, solvent precipitation method, double emulsion/solvent evaporation technique, particle replication in nonwetting templates, and lyophilization. The resulting polydisperse mixtures can be refined further by specialized milling techniques. Jet-milling of drugs and excipients under nitrogen gas with a nano-jet milling machine is one non-limiting exemplary method known in the art for creating nanoparticles meant for pulmonary drug delivery.

### Exemplary Applications

The disclosure provides materials and methods for delivery of an antiviral peptide (and, optionally, one or more complementary agents) to respiratory tract for the purposes of treating, preventing, reducing the likelihood of having, reducing the severity of and/or slowing the progression of a medical condition in a subject. In a preferred embodiment, the medical condition is a viral respiratory infection, or exposure to a virus.

In some cases, a subject in need of treatment for a disease or disorder disclosed herein, such as an infectious disease, is symptomatic for the disease or disorder. In some cases, a subject in need of treatment for a disease or disorder disclosed herein, such as an infectious disease, is asymptomatic for the disease or disorder. A subject in need of treatment for a disease or disorder disclosed herein can be identified by a skilled practitioner, such as without limitation, a medical doctor or a nurse.

Influenza spreads around the world in seasonal epidemics, resulting in the deaths of hundreds of thousands annually-millions in pandemic years. For example, three influenza pandemics occurred in the 20th century and killed tens of millions of people, with each of these pandemics being caused by the appearance of a new strain of the virus in humans. Often, these new strains result from the spread of an existing influenza virus to humans from other animal species. Influenza viruses are RNA viruses of the family Orthomyxoviridae, which comprises five genera: Influenza virus A, Influenza virus B, Influenza virus C, Isavirus, and Thogoto virus. The influenza A virus can be subdivided into different serotypes based on the antibody response to these viruses. The serotypes that have been confirmed in humans, ordered by the number of known human pandemic deaths, are: H1N1 (the strain that caused Spanish influenza in 1918), H2N2 (caused Asian Influenza in 1957), H3N2 (caused Hong Kong Flu in 1968), H5N1 (a pandemic threat in the 2007-08 influenza season), H7N7 (has unusual zoonotic potential), H1N2 (endemic in humans and pigs), H9N2, H7N2, H7N3 and H10N7. Influenza B causes seasonal flu and influenza C causes local epidemics, and both influenza B and C are less common than influenza A.

Coronaviruses are a family of common viruses that cause a range of illnesses in humans from the common cold to severe acute respiratory syndrome (SARS). Coronaviruses can also cause a number of diseases in animals. Coronaviruses are enveloped, positive-stranded RNA viruses whose name derives from their characteristic crown-like appearance in electron micrographs. Coronaviruses are classified as a family within the Nidovirales order, viruses that replicate using a nested set of mRNAs. The coronavirus subfamily is further classified into four genera: alpha, beta, gamma, and delta coronaviruses. The human coronaviruses (HCoVs) are in two of these genera: alpha coronaviruses (including HCoV-229E and HCoV-NL63) and beta coronaviruses (including HCoV-HKU1, HCoV-0043, Middle East respiratory syndrome coronavirus (MERS-CoV), the severe acute respiratory syndrome coronavirus (SARS-CoV and SARS-CoV-2).

A number of other respiratory viruses are well known in the art, such as described by Nichols et al. (23), included herein in its entirety.

The disclosure contemplates veterinary application of the materials and methods described herein, involving all mammals, including, but not limited to dogs, cats, horses, pigs, sheep, goats, and cows.

In one embodiment of the disclosure, the system serves multiple purposes, where more than one medical condition is targeted simultaneously. An example of such a multipurpose drug delivery system involves the treatment of a SARS-CoV-2 infection along with concomitant dampening of the host immune response.

### EQUIVALENTS

The present disclosure is not to be limited in terms of the particular embodiments described in this application, which are intended as illustrations of various aspects. Many modifications and variations can be made without departing from the spirit and scope of the disclosure, as will be apparent to those skilled in the art. Functionally equivalent methods, systems, and apparatus within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting. As will be understood by one skilled in the art, for any and all purposes, such as in terms of providing a written description, all ranges disclosed herein also encompass any and all possible subranges and combinations of subranges thereof.

Many changes can be made to the preferred embodiments of the invention without departing from the scope thereof. It is intended that all matter contained herein be considered illustrative of the invention and not in a limiting sense.

While various aspects and embodiments have been disclosed herein, other aspects and embodiments will be apparent to those skilled in the art. All references cited herein are incorporated by reference in their entireties.

### EXAMPLES

### Example 1—Formulation 1 (Pulmonary Administration)

For aerosol administration, Peptide 346-001 is used as a liquid solution along with a surfactant and propellant. Typical percentages of Peptide 346-001 are 0.01%-20% w/w, preferably 1%-10% w/w. The surfactant is nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% w/w of the composition, preferably 0.25-5% w/w. The balance of the composition is ordinarily propellant. A carrier can also be included as desired, e.g., lecithin for intranasal delivery.

### Example 2—Formulation 2 (Nasal Administration)

Aqueous solutions of Peptide 346-001 (concentrations shown in Example 1) and bulking agents (mannitol or lactose) are prepared in Dulbecco's phosphate-buffered saline (pH 8.0, adjusted with aqueous ammonia, 28% w/w). The resulting solutions are filtered through a 0.2 μm PES filter (Celltreat Scientific Products, Shirley, Mass.) and aliquots (2 mL) of the solution are filled into sterile 5 mL borosilicate glass vials. Rubber stoppers are placed on top of the vials prior lyophilization. At the completion of the cycle, nitrogen is introduced into the chamber. Vials are sealed with rubber stoppers and aluminum crimp-top closure.

### Example 3—In Vitro SARS-CoV-2 Prevention Model (Study 1)

Dosing concentrations of Peptide 346-001 and Peptide 346-009 (negative control) were prepared from DMSO stock solutions by dilution with 1×MEM (Gibco cat #11095-080; Lot 2192695) at predetermined dilution levels. The resulting dosing solutions (100 μL) were added in triplicate to freshly aspirated wells containing 80% confluent VERO E6 cultures, prepared in 48-well format (18 wells were used for each compound). Three wells of the 18 were treated with the highest concentration, but were not infected with virus to serve as toxicity controls. The remaining wells (12) including four used to test toxicity of the DMSO vehicle (0.5% stock in MEM medium, 100 μL/well) without infection; four wells similarly treated and then infected to determine impact of the DMSO on the virus; and 4 wells that were not treated (provided 100 μL of medium) and infected to show maximal viral infection for the study.

The treated plate was transferred to the BLS3 facility where the designated wells were challenged with 10⁴ TCID₅₀ SARS-CoV-2 (2019-nCoV/USA-WA1/2020 strain) in 100 μL of serum-free MEM (Gibco). The time between drug and virus addition to the wells was ca. 20 min. The plate was incubated for 48 h at 37° C. Each well was then lysed by addition of 200 μL of MagNAPure External Lysis solution (Roche cat number 06374913001; LOT 35850400) without aspiration. The 4004 of lysed material was subjected to automated MagNAPure96 IVD DNA and viral NA extraction method. Quantification of viral titers was completed using RT-qPCR assays optimized for clinical diagnostics. For each sample, copies of the SARS-CoV-2 envelope “E” gene, open reading frame 8 “orf8” gene and the human RNAseP “RP” gene were quantified on CFX Real-Time Systems (Bio-Rad), or equivalent. Primer sequences and amplimer specifics are provided below. The two viral targets indicated impact on viral infection while the RP copy number was a surrogate for cellular toxicity.

E Amplimer: 113 bp (forward and reverse primers in
bold)
(SEQ ID NO: 74)
ACAGGTACGTTAATAGTTAATAGCGT-
ACTTCTTTTTCTTGCTTTCGTGGTATTCTTGCTAGTTACACTAGCCATCC
TTACTGCGCTTCGATTG-TGTGCGTACTGCTGCAATAT
Base pairs: 26277 to 26389;
Sequence ID: LC528233.1
orf 8 Amplimer: 91 bp (forward and reverse primers
in bold)
(SEQ ID NO: 75)
AATCAGCACCTTTAATTGAATTG-
TGCGTGGATGAGGCTGGTTCTAAATCACCCATTCAGTACATCGA-
TATCGGTAATTATACAGTTTCCTG
Base pairs: 28059 to 28149;
Sequence ID: LC528233.1
RNAse P Amplimer: 64 bp (forward and reverse
primers in bold)
(SEQ ID NO: 76)
AGA TTT GGA CCT GCG AGC G
(SEQ ID NO: 77)
GAG CGG CTG TCT CCA CAA GT
Base pairs: 28 to 92; GenBank Sequence
ID: NM_006413.5

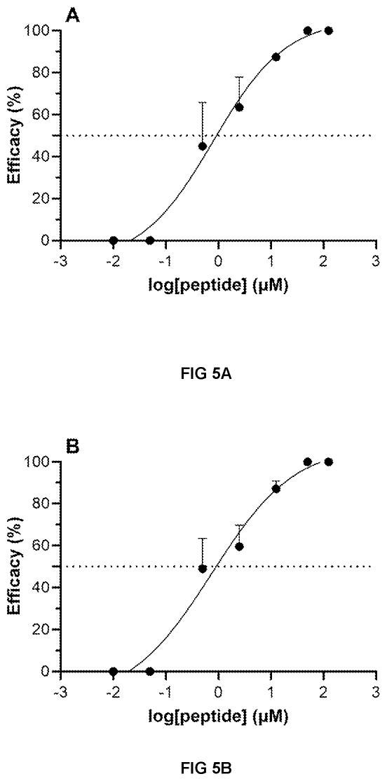

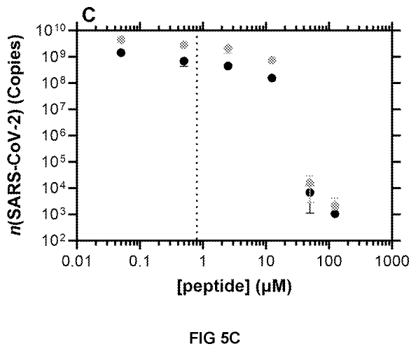

Results from this study are shown in FIGS. 5A-5C. Under these experimental conditions, the antiviral peptide displayed a surprising, high potency against SARS-CoV-2 infection. At 50 μM dosing concentration, a reduction on viral copy numbers of >5 orders of magnitude was observed, and >7 orders of magnitude at 125 μM dosing concentration (FIG. 5C).

### Example 4—In Vitro SARS-CoV-2 Prevention Model (Study 2)

Eleven dosing concentrations of Peptides 346-001, 346-002, 346-003, 346-004, 346-005, 346-007, 346-008, and 346-009 (negative control) were prepared as freshly created DMSO stock solutions by dilution with 1×MEM (Gibco cat #11095-080; Lot 2192695) at predetermined dilution levels. The resulting dosing solutions (50 μL) were added in quadruplicate to freshly aspirated wells containing 60% confluent VERO E6 cultures, prepared in 96-well format (44 wells were used for each compound). A final 4 wells were not treated (50 μL MEM) serving as maximal viral infection controls.

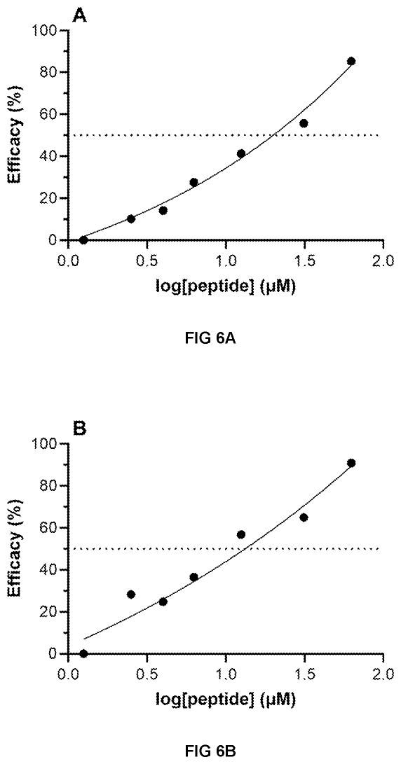

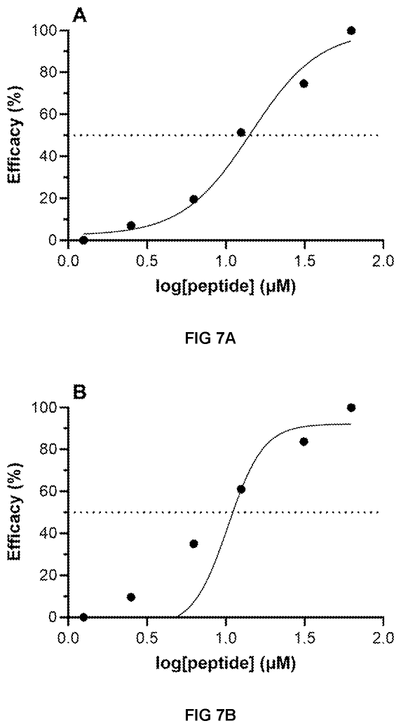

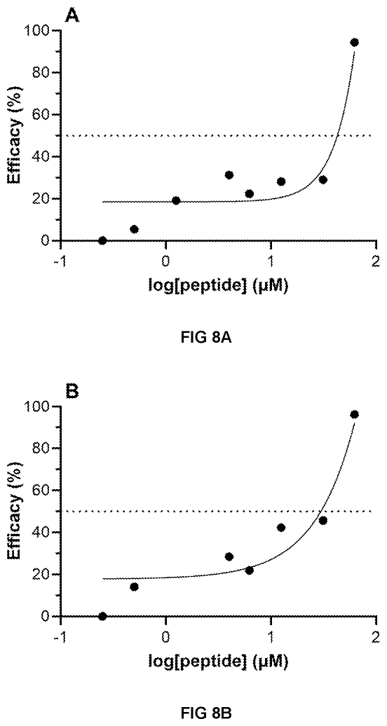

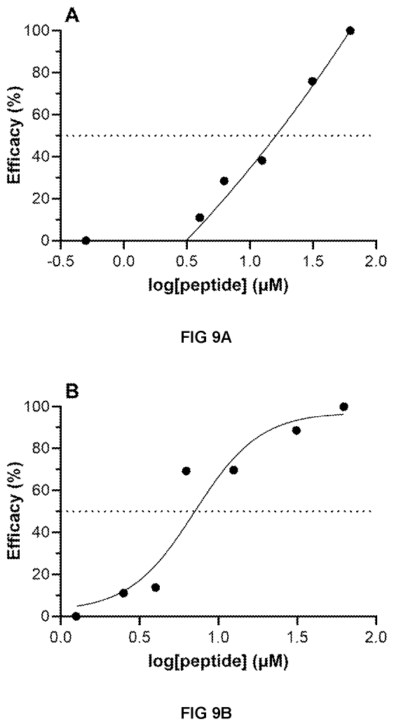

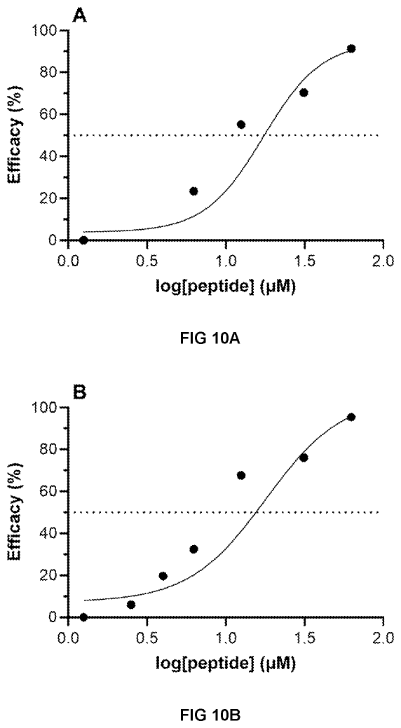

The treated plates were transferred to the BLS3 facility where the designated wells were challenged with 10³ TCID₅₀ SARS-CoV-2 (2019-nCoV/USA-WA1/2020 strain) in 504 of serum-free MEM (Gibco). The time between drug and virus addition to the wells was ca. 40 min. The plates were incubated for 48 h at 37° C. Each well was then lysed by addition of 100 μL of MagNAPure External Lysis solution (Roche cat number 06374913001; LOT 35850400) without aspiration. The 2004 of lysed material was subjected to automated MagNAPure96 IVD DNA and viral NA extraction method. Quantification of viral titers was completed as described in EXAMPLE 3. Results from this study are shown in FIG. 6A-10B. Comparison of different antiviral peptides based on SEQ. ID 346-001 afforded surprising results in terms of the dose-response relationships against SARS-CoV-2. The potencies of the peptides tested varied depending on the sequence, but all provided robust activity against SARS-CoV-2. The shapes of the dose-response curves varied substantially, even between peptides of identical sequences, but using L-(SEQ. ID 346-001) versus D-(SEQ. ID 346-002) amino acids, as shown in FIGS. 6A/6B and 7A/7B, respectively. Peptide SEQ. ID 346-003 displayed an unexpected and unusual dose response curve shape (FIGS. 8A and 8B).

### Example 5—In Vitro SARS-CoV-2 Prevention Model (Study 3)

Eleven dosing concentrations of Peptides 346-001, 346-002, 346-004, and 346-007 were prepared as freshly created DMSO stock solutions by dilution with 1×MEM (Gibco cat #11095-080; Lot 2192695) at predetermined dilution levels. The resulting dosing solutions (50 μL) were added in quadruplicate to freshly aspirated wells containing 80% confluent VERO E6 cultures, prepared in 96-well format (44 wells were used for each compound). A final 4 wells were not treated (50 μL MEM) serving as maximal viral infection controls.

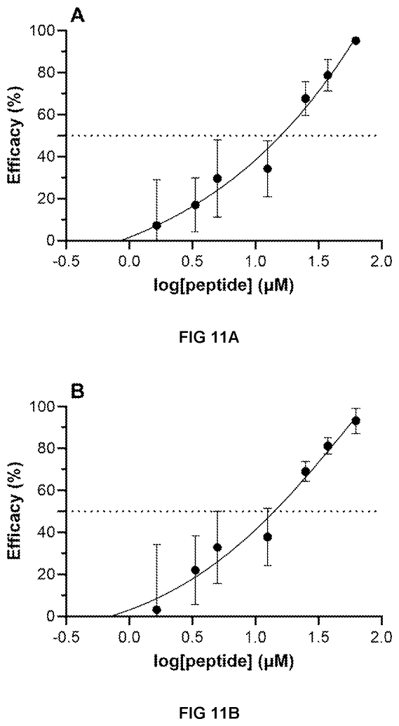

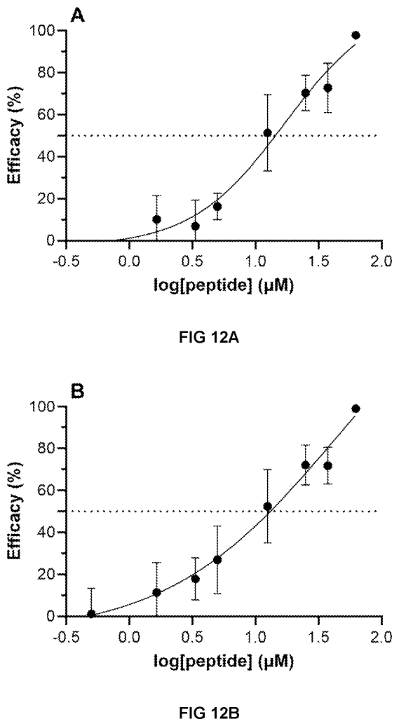

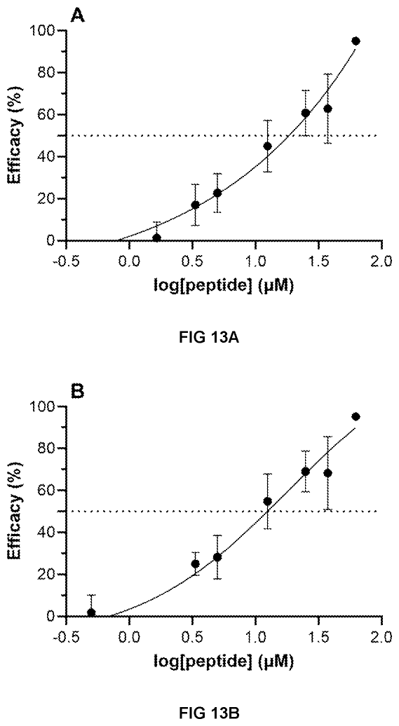

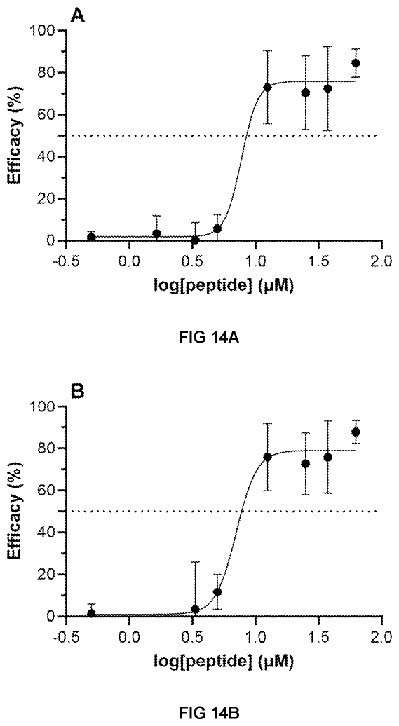

The treated plates were transferred to the BLS3 facility where the designated wells were challenged with 10³ TCID₅₀ SARS-CoV-2 (2019-nCoV/USA-WA1/2020 strain) in 504 of serum-free MEM (Gibco) for VERO E6 wells. The time between drug and virus addition to the wells was ca. 40 min. The plates were incubated for 48 h at 37° C. Each well was then lysed by addition of 1004 of MagNAPure External Lysis solution (Roche cat number 06374913001; LOT 35850400) without aspiration. The 200 μL of lysed material was subjected to automated MagNAPure96 IVD DNA and viral NA extraction method. Quantification of viral titers was completed as described for EXAMPLE 3. Results from this study are shown in FIG. 11A-14B. Comparison of different antiviral peptides based on SEQ. ID 346-001 afforded surprising results in terms of the dose-response relationships against SARS-CoV-2. All of the peptides tested provided robust activity against SARS-CoV-2. All peptides tested except SEQ. ID 346-007 displayed similar dose-response relationships and antiviral potencies. However, SEQ. ID 346-007 led to a steep dose response relationship with ca. double the potency of the other peptides tested in this example (FIGS. 14A and 14B).

### Example 6—In Vitro SARS-CoV-2 Prevention Model (Study 4)

Eleven dosing concentrations of Peptides 346-001, 346-002, 346-004, and 346-007 were prepared as freshly created DMSO stock solutions by dilution with 1×MEM (Gibco cat #11095-080; Lot 2192695) at predetermined dilution levels. The resulting dosing solutions (5 μL) were added in quadruplicate to air-liquid interfaced human nasal epithelial cells (NEC) Transwell cultures, prepared in 96-well format (44 wells were used for each compound).

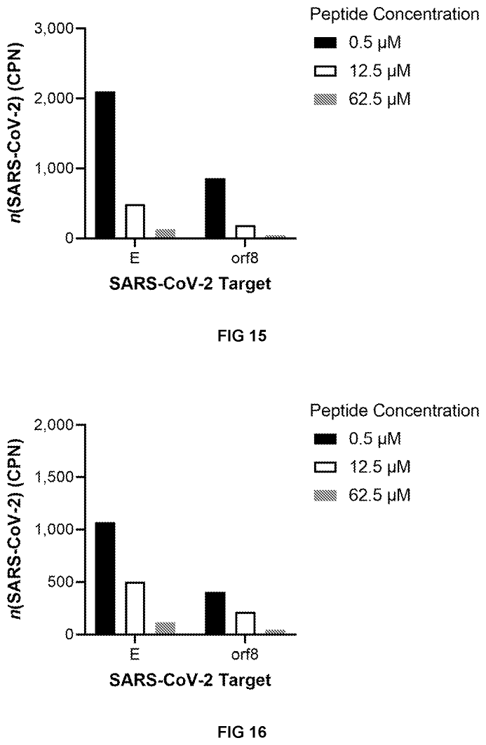

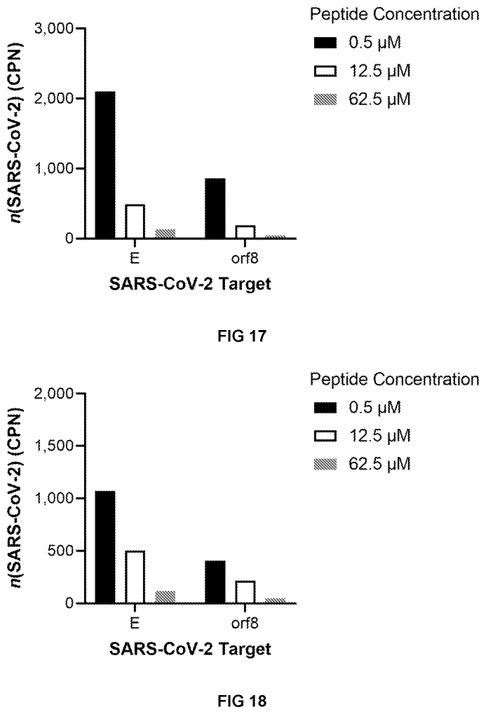

The treated plates were transferred to the BLS3 facility where the designated wells were challenged with 10³ TCID₅₀ SARS-CoV-2 (2019-nCoV/USA-WA1/2020 strain) in 5 μL of serum-free MEM (Gibco). The time between drug and virus addition to the wells was ca. 40 min. The plates were incubated for 48 h at 37° C. Each well was then lysed by addition of 200 μL of MagNAPure External Lysis solution (Roche cat number 06374913001; LOT 35850400) in the apical chamber. The 200 μL of lysed material was subjected to automated MagNAPure96 IVD DNA and viral NA extraction method. Quantification of viral titers was completed as described for Study 1. Results from this study are shown in FIG. 15-18. An innovative human nasal epithelial culture system with an apical air interface was used in lieu of VERO cells (i.e., non-human primate origin) in this example to provide a more real-world evaluation of select antiviral peptides. Surprisingly, the potency of all peptides tested was under the 12.5 μM dosing concentration.

### Example 7—Substitution Analysis

An alanine scan was performed on Peptide 346-001 to identify amino acids in the sequence that are important for virucidal efficacy. An established HIV-1 in vitro model was used in the first round of screening. Peptide 346-001 is effective against HIV-1 and respiratory viruses, with similar in vitro EC₅₀ values.

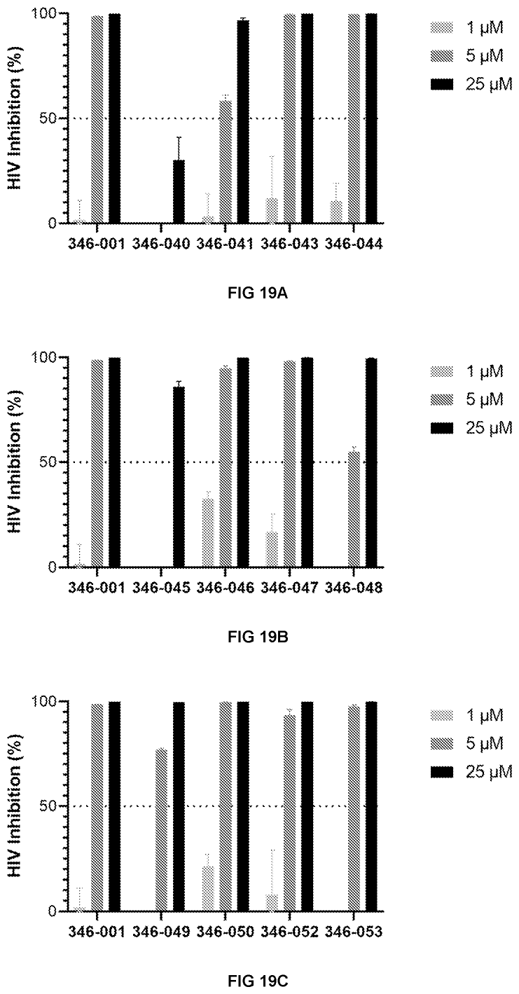

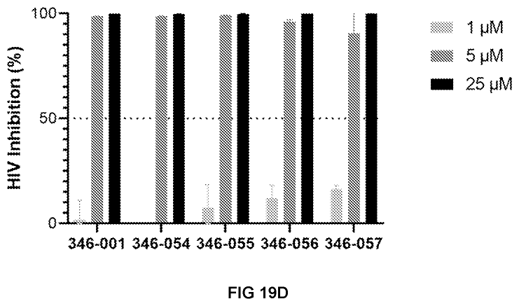

HeLa TZM reporter cells (expressing CD4, CCR4, CCR5, and β-galactosidase) were grown to 100,000 cells in a 96-well format, in triplicate. An HIV-1 (primary R5 JR-CSF) stock was grown in human peripheral blood mononuclear cells (PBMCs) and standardized by HIV-1 p24 ELISA. The peptide solution (10 μL, diluted from a DMSO stock) and HIV-1 aliquot (10 μL) were mixed and incubated at 37° C. for 0.5 h. Cells were treated with the peptide-virus mixture (20 μL), or control, in triplicate at each dose by mixing with media (150 μL). The culture was incubated for 6 h at 37° C. and then washed. The challenge endpoint was at 48 h, and viral infection was analyzed in supernatant by β-galactosidase quantification. Negative controls consisted of the most concentrated DMSO solution used above (no drug). Positive control consisted of Peptide 346-001, prepared as above. The results are shown in FIG. 19A-19D and illustrate that Peptide 346-001 tolerates substitutions and retains antiviral activity.

TABLE 1
Examples of peptides against respiratory viruses.

| | SEQ | |
|---|---|---|
| | ID | |
| Peptide | NO: | Peptide Sequence and Modifications |
| 346-001 | 1 | SWLRDIWDWICEVLSDFK |
| 346-002 | 2 | swlrdiwdwicevlsdfk |
| 346-003 | 3 | GSWLRDIWDWICEVLSDFK |
| 346-004 | 4 | SWLRDIWDWICEVLSDFKTW |
| 346-005 | 5 | kfdslveciwdwidrlws |
| 346-006 | 6 | KWLCRIWSWISDVLDDFE |
| 346-007 | 7 | SIWRDWVDLICEFLSDWK |
| 346-008 | 8 | DWLRIIWDWVCSVVSDFK |
| 346-009 | 9 | PLKPTKRSFIKDLLFNKV |
| 346-010 | 10 | SWLRDIWDWISEVLSDFK |
| 346-011 | 11 | swlrdiwdwisevlsdfk |
| 346-012 | 12 | SWLRDIWDWICEVLSDFR |
| 346-013 | 13 | SYLRDIWDYICEVLSDFK |
| 346-014 | 14 | SYLRDIWDYISEVLSDFK |
| 346-015 | 15 | SYLREIWDYISEVLSDFR |
| 346-016 | 16 | SWLREIWDWICEVLSDFK |
| 346-017 | 17 | Ac-SWLRDIWDWICEVLSDFK-NH₂ |
| 346-018 | 18 | Ac-swlrdiwdwicevlsdfk-NH₂ |
| 346-019 | 19 | Ac-GSWLRDIWDWICEVLSDFK-NH₂ |
| 346-020 | 20 | Ac-SWLRDIWDWICEVLSDFKTW-NH₂ |
| 346-021 | 21 | Ac-kfdslveciwdwidrlws-NH₂ |
| 346-022 | 22 | Ac-KWLCRIWSWISDVLDDFE-NH₂ |
| 346-023 | 23 | Ac-SIWRDWVDLICEFLSDWK-NH₂ |
| 346-024 | 24 | Ac-DWLRIIWDWVCSVVSDFK-NH₂ |
| 346-025 | 25 | Ac-SWLRDIWDWISEVLSDFK-NH₂ |
| 346-026 | 26 | Ac-swlrdiwdwisevlsdfk-NH₂ |
| 346-027 | 27 | Ac-SWLRDIWDWICEVLSDFR-NH₂ |
| 346-028 | 28 | Ac-SYLRDIWDYICEVLSDFK-NH₂ |
| 346-029 | 29 | Ac-SYLRDIWDYISEVLSDFK-NH₂ |
| 346-030 | 30 | Ac-SYLREIWDYISEVLSDFR-NH₂ |
| 346-031 | 31 | Ac-SWLREIWDWICEVLSDFK-NH₂ |
| 346-032 | 32 | SWLRDIWDWISEVLSDFR |
| 346-033 | 33 | SWLRDIWDYISEVLSDFR |
| 346-034 | 34 | swlrdiwdyisevlsdfr |
| 346-035 | 35 | SWLRDIWDYICEVLSDFR |
| 346-036 | 36 | Ac-SWLRDIWDWISEVLSDFR-NH₂ |
| 346-037 | 37 | Ac-SWLRDIWDYISEVLSDFR-NH₂ |
| 346-038 | 38 | Ac-swlrdiwdyisevlsdfr-NH₂ |
| 346-039 | 39 | Ac-SWLRDIWDYICEVLSDFR-NH₂ |
| 346-040 | 40 | AWLRDIWDWICEVLSDFK |
| 346-041 | 41 | SALRDIWDWICEVLSDFK |
| 346-042 | 42 | SWARDIWDWICEVLSDFK |
| 346-043 | 43 | SWLADIWDWICEVLSDFK |
| 346-044 | 44 | SWLRAIWDWICEVLSDFK |
| 346-045 | 45 | SWLRDAWDWICEVLSDFK |
| 346-046 | 46 | SWLRDIADWICEVLSDFK |
| 346-047 | 47 | SWLRDIWAWICEVLSDFK |
| 346-048 | 48 | SWLRDIWDAICEVLSDFK |
| 346-049 | 49 | SWLRDIWDWACEVLSDFK |
| 346-050 | 50 | SWLRDIWDWIAEVLSDFK |
| 346-051 | 51 | SWLRDIWDWICAVLSDFK |
| 346-052 | 52 | SWLRDIWDWICEALSDFK |
| 346-053 | 53 | SWLRDIWDWICEVASDFK |
| 346-054 | 54 | SWLRDIWDWICEVLADFK |
| 346-055 | 55 | SWLRDIWDWICEVLSAFK |
| 346-056 | 56 | SWLRDIWDWICEVLSDAK |
| 346-057 | 57 | SWLRDIWDWICEVLSDFA |
| 346-058 | 58 | Ac-SWLRDIWDWICEVLSDFKK |
| 346-059 | 59 | KSWLRDIWDWICEVLSDFK-NH₂ |
| 346-060 | 60 | Ac-K(palmitoyl)-SWLRDIWDWICEVLSDFK-NH₂ |
| 346-061 | 61 | Ac-SWLRDIWDWICEVLSDFK-K(palmitoyl)-NH₂ |
| 346-062 | 62 | Ac-SWLRDIWDWICEVLSDFK-amino-PEG12- |
| | | propionyl-NH₂ |
| 346-063 | 63 | Ac-amino-PEG12-propionyl- |
| | | SWLRDIWDWICEVLSDFK-NH₂ |
| 346-064 | 64 | Ac-SWLRDIWDWICEVLSDFKC(succinimido- |
| | | propionylaminoethyl-mPEG2K)-NH₂ |
| 346-065 | 65 | Ac-C(succinimido-propionylaminoethyl- |
| | | mPEG2K)-SWLRDIWDWICEVLSDFK-NH₂ |
| 346-066 | 66 | H-K(oleoyl)-SWLRDIWDWICEVLSDFK-NH₂ |
| 346-067 | 67 | Ac-SWLRDIWDWICEVLSDFK-K(oleoyl)-NH₂ |
| 346-068 | 68 | H-C(cholesteryloxycarbonylmethyl)- |
| | | SWLRDIWDWICEVLSDFK-NH₂ |
| 346-069 | 69 | Ac-SWLRDIWDWICEVLSDFK- |
| | | C(cholesteryloxycarbonylmethyl)-NH₂ |
| 346-070 | 70 | Ac-K(succinyl-D-α-tocopherol)- |
| | | SWLRDIWDWICEVLSDFK-NH₂ |
| 346-071 | 71 | Ac-SWLRDIWDWICEVLSDFK-K(succinyl-D-α- |
| | | tocopherol)-NH₂ |

Upper case single letter amino acid abbreviations indicate L-isomer; lower case single letter amino acid abbreviations indicate D-isomer.

TABLE 2
Examples of general peptide conjugates based on
Peptide 346-001 backbone.

Conj-Ser-Trp-Leu-Arg-Asp-Ile-Trp-Asp-Trp-Ile-Cys-
Glu-Val-Leu-Ser-Asp-Phe-Lys-NH₂
Ac-Ser-Trp-Leu-Arg-Asp-Ile-Trp-Asp-Trp-Ile-Cys-
Glu-Val-Leu-Ser-Asp-Phe-Lys-Conj
Conj-Ser-Trp-Leu-Arg-Asp-Ile-Trp-Asp-Trp-Ile-Cys-
Glu-Val-Leu-Ser-Asp-Phe-Lys-Conj
“Conj” denotes conjugate

All references cited herein are incorporated by reference in their entirety as though fully set forth herein. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (Boca Raton, Fla., 2008); Oxford Textbook of Medicine, Oxford Univ. Press (Oxford, England, UK, May 2010, with 2018 update); Harrison's Principles of Internal Medicine, Vol 0.1 and 2, 20th ed., McGraw-Hill (New York, N.Y., 2018); Singleton and Sainsbury, Dictionary of Microbiology and Molecular Biology, 3^{rd} ed., revised ed., J. Wiley & Sons (New York, N.Y., 2006); Smith, March's Advanced Organic Chemistry Reactions, Mechanisms and Structure 7^{th} ed., J. Wiley & Sons (New York, N.Y., 2013); and Singleton, Dictionary of DNA and Genome Technology, 3^{rd} ed., Wiley-Blackwell (Hoboken, N.J., 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. The entire document is intended to be related as a unified disclosure, and it should be understood that all combinations of features described herein (even if described in separate sections) are contemplated, even if the combination of features is not found together in the same sentence, or paragraph, or section of this document.

It should be understood that, while various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” or “consist essentially of” the feature. The term “a” or “an” refers to one or more, and the terms “a” (or “an”), “one or more,” and “at least one” can be used interchangeably herein. The term “or” should be understood to encompass items in the alternative or together, unless context unambiguously requires otherwise.

### REFERENCES CITED

1. Le et al., The COVID-19 Vaccine Development Landscape. Nat. Rev. Drug Discov. 19, 305-306.
2. Baric, R. S. In Virology of Coronaviruses, 2020 Conference on Retroviruses and Opportunistic Infections (CR₀₁), Boston, Mass., Mar. 8-11, 2020; CROI, Alexandria, Va.: Boston, Mass., 2020; p Abstract Number 2008.
3. Peeples, L., News Feature: Avoiding Pitfalls in the Pursuit of a COVID-19 Vaccine. Proc. Natl. Acad. Sci. USA 2020, 117 (15), 8218-8221.
4. Tay et al., The Trinity of COVID-19: Immunity, Inflammation and Intervention. Nat. Rev. Immunol. 2020.
5. Bobardt et al., Hepatitis C Virus NS5A Anchor Peptide Disrupts Human Immunodeficiency Virus. Proc. Natl. Acad. Sci. U.S.A 2008, 105 (14), 5525-5530.
6. Cheng et al., A Virocidal Arnphipathic Alpha-helical Peptide that Inhibits Hepatitis C Virus Infection in Vitro. Proc. Natl. Acad. Sci. U.S.A 2008, 105 (8), 3088-3093.
7. Kolb, H. C.; Finn, M. G.; Sharpless, K. B., Click Chemistry: Diverse Chemical Function from a Few Good Reactions. Angew. Chem. Int. Ed. Engl. 2001, 40 (11), 2004-2021.
8. Kolb, H. C.; Sharpless, K. B., The Growing Impact of Click Chemistry on Drug Discovery. Drug Discov. Today 2003, 8 (24), 1128-1137.
9. Meldal, M.; Tornøe, C. W., Cu-catalyzed Azide-alkyne Cycloaddition. Chem. Rev. 2008, 108 (8), 2952-3015.
10. Moses, J. E.; Moorhouse, A. D., The Growing Applications of Click Chemistry. Chem. Soc. Rev. 2007, 36 (8), 1249-62.
11. Bundgaard, H., The Utility of the Prodrug Approach to Improve Peptide Absorption. J. Control. Release 1992, 21 (1-3), 63-72.
12. Oliyai, R.; Stella, V. J., Prodrugs of Peptides and Proteins for Improved Formulation and Delivery. Annu. Rev. Pharmacol. Toxicol. 1993, 33, 521-544.
13. Agarwa et al., Peptide Prodrugs: Improved Oral Absorption of Lopinavir, a HIV Protease Inhibitor. Int. J. Pharm. 2008, 359 (1-2), 7-14.
14. Nchioua et al., The Zinc Finger Antiviral Protein Restricts SARS-CoV-2. bioRxiv 2020.
15. Wang et al., Peptide-drug Conjugates as Effective Prodrug Strategies for Targeted Delivery. Adv. Drug Deliv. Rev. 2017, 110-111, 112-126.
16. Djupesland, P. G., Nasal Drug Delivery Devices: Characteristics and Performance in a Clinical Perspective-A Review. Drug Deliv. Transl. Res. 2013, 3 (1), 42-62.
17. Patil et al., Pulmonary Drug Delivery Strategies: A Concise, Systematic Review. Lung India 2012, 29 (1), 44-9.
18. Ibrahim et al., Inhalation Drug Delivery Devices: Technology Update. Med. Devices (Auckl) 2015, 8, 131-139.
19. Moon et al., Delivery Technologies for Orally Inhaled Products: an Update. AAPS PharmSciTech 2019, 20 (3), 117.
20. Chang et al., Mechanisms of Protein Stabilization in the Solid State. J. Pharm. Sci. 2009, 98 (9), 2886-2908.
21. Mensink et al., How Sugars Protect Proteins in the Solid State and During Drying (Review): Mechanisms of Stabilization in Relation to Stress Conditions. Eur. J. Pharm. Biopharm. 2017, 114, 288-295.
22. Forney-Stevens et al., Addition of Amino Acids to Further Stabilize Lyophilized Sucrose-Based Protein Formulations: I. Screening of 15 Amino Acids in Two Model Proteins. J. Pharm. Sci. 2015, 105, 697-704.
23. Nichols et al., Respiratory Viruses Other Than Influenza Virus: Impact and Therapeutic Advances. Clin. Microbiol. Rev. 2008, 21 (2), 274-290.
24. Beigel et al., Remdesivir for the Treatment of Covid-19—Preliminary Report. N. Engl. J. Med. 2020.

What is claimed is:

1. A method of treating or preventing a viral respiratory disease, the method comprising administering to a subject in need thereof a peptide comprising the amino acid sequence of Peptide 346-001 or variant thereof comprising one, two, or three amino acid substitutions, wherein the administration is via intranasal or pulmonary routes of administration.

2. The method of claim 1, wherein the peptide comprises the amino acid sequence of Peptide 346-001.

3. The method of claim 1, wherein the peptide comprises the amino acid sequence of Peptide 346-001 having one, two, or three amino acid substitutions, the substitutions being selected from the following: the S at position 1 is substituted with K, D, A, N, M, T, or V; the W at position 2 is substituted with I, Y, F, P, L, V, H, M, A, T, or S; the L at position 3 is substituted with W, I, M, V, F, M, A, or T; the R at position 4 is substituted with C, K, Q, H, P, or C; the D at position 5 is substituted with R, I, E, N, or Y; the I at position 6 is substituted with W, V, L, or F; the W at position 7 is substituted with V, Y, F, P, L, V, H, or D; the D at position 8 is substituted with S, E, N, Y, or S; the W at position 9 is substituted with L, Y, F, P, L, V, or H; the I at position 10 is substituted with V, L, or F; the C at position 11 is substituted with S, A, P, M, H, or T; the E at position 12 is substituted with D, S, Q, Y, or T; the V at position 13 is substituted with F, I, L, A, F, or M; the L at position 14 is substituted with V, I, M, or F; the S at position 15 is substituted with D, A, N, T, Y, or P; the D at position 16 is substituted with E, N, Y, or S; the F at position 17 is substituted with W, Y, L, P, or H; or the K at position 18 is substituted with E, R, H, R, P, or Q.

4. The method of any one of claims 1-3, wherein the peptide is modified with a lipophilic moiety via a linker bonded to a lysine, serine, or cysteine of the peptide or variant.

5. The method of any one of claims 1 to 4, wherein the viral respiratory disease is a coronavirus infection, an influenza infection, or a respiratory syncytial virus (RSV) infection.

6. The method of any one of claims 1 to 5, wherein the viral respiratory disease is a coronavirus infection.

7. The method of any one of claims 1 to 5, wherein the viral respiratory disease is an influenza infection.

8. The method of any one of claims 1 to 5, wherein the viral respiratory disease is a respiratory syncytial virus (RSV) infection.

9. The method of any one of claims 1 to 6, wherein the viral respiratory disease is SARS-CoV-2 infection.

10. The method of any one of claims 1 to 9, further comprising administering one or more complementary agents.

11. A method of treating or preventing a viral respiratory disease, the method comprising administering to a subject in need thereof a peptide comprising
X₁-X₂-X₃-X₄-X₅-X₆-X₇-X₈-X₉-X₁₀-X₁₁-X₁₂-X₁₃-X₁₄-X₁₅-X₁₆-X₁₇-X₁₈ (Formula 1), wherein X, is S, K, D, A, N, M, T, or V; X₂ is W, I, Y, F, P, L, V, H, M, A, T, or S; X₃ is L, W, I, M, V, F, M, A, or T; X₄ is R, C, K, Q, H, P, or C; X₅ is D, R, I, E, N, or Y; X₆ is I, W, V, L, or F; X₇ is W, V, Y, F, P, L, V, H, or D; X₈ is D, S, E, N, Y, or S; X₉ is W, L, Y, F, P, L, V, or H; X₁₀ is I, V, L, or F; X₁₁ is C, S, A, P, M, H, or T; X₁₂ is E, D, S, Q, Y, or T; X₁₃ is V, F, I, L, A, F, or M; X₁₄ is L, V, I, M, or F; X₁₅ is S, D, A, N, T, Y, or P; X₁₆ is D, E, N, Y, or S; X₁₇ is F, W, Y, L, P, or H; and X₁₈ is K, E, R, H, R, P, or Q, wherein the administration is via intranasal or pulmonary routes of administration.

12. The method of claim 11, wherein the peptide comprises an amino acid sequence set forth in Table 1.

13. The method of claim 11 or claim 12, wherein the viral respiratory disease is a coronavirus infection, an influenza infection, or a respiratory syncytial virus (RSV) infection.

14. The method of claim 13, wherein the viral respiratory disease is a coronavirus infection.

15. The method of claim 13, wherein the viral respiratory disease is an influenza infection.

16. The method of claim 13, wherein the viral respiratory disease is SARS-CoV-2 infection.

17. The method of any one of claims 11 to 16, wherein the viral respiratory disease is SARS-CoV-2 infection.

18. The method of any one of claims 11 to 17, further comprising administering one or more complementary agents.

19. The method of any one of claims 11 to 18, wherein the peptide is modified with a lipophilic moiety via a linker bonded to a lysine, serine, or cysteine of the peptide or variant.

20. The method of any one of claims 11 to 19, wherein X₁₁ is not C.

21. The method of any one of claims 11 to 20, wherein one or more amino acids are D-amino acids.


SEQUENCE LISTING (78 sequences)

SEQ ID NO: 1 - protein, 18 aa, Artificial Sequence
  Synthetic
SWLRDIWDWICEVLSDFK

SEQ ID NO: 2 - protein, 18 aa, Artificial Sequence
  Synthetic
SWLRDIWDWICEVLSDFK

SEQ ID NO: 3 - protein, 19 aa, Artificial Sequence
  Synthetic
GSWLRDIWDWICEVLSDFK

SEQ ID NO: 4 - protein, 20 aa, Artificial Sequence
  Synthetic
SWLRDIWDWICEVLSDFKTW

SEQ ID NO: 5 - protein, 18 aa, Artificial Sequence
  Synthetic
KFDSLVECIWDWIDRLWS

SEQ ID NO: 6 - protein, 18 aa, Artificial Sequence
  Synthetic
KWLCRIWSWISDVLDDFE

SEQ ID NO: 7 - protein, 18 aa, Artificial Sequence
  Synthetic
SIWRDWVDLICEFLSDWK

SEQ ID NO: 8 - protein, 18 aa, Artificial Sequence
  Synthetic
DWLRIIWDWVCSVVSDFK

SEQ ID NO: 9 - protein, 18 aa, Artificial Sequence
  Synthetic
PLKPTKRSFIKDLLFNKV

SEQ ID NO: 10 - protein, 18 aa, Artificial Sequence
  Synthetic
SWLRDIWDWISEVLSDFK

SEQ ID NO: 11 - protein, 18 aa, Artificial Sequence
  Synthetic
SWLRDIWDWISEVLSDFK

SEQ ID NO: 12 - protein, 18 aa, Artificial Sequence
  Synthetic
SWLRDIWDWICEVLSDFR

SEQ ID NO: 13 - protein, 18 aa, Artificial Sequence
  Synthetic
SYLRDIWDYICEVLSDFK

SEQ ID NO: 14 - protein, 18 aa, Artificial Sequence
  Synthetic
SYLRDIWDYISEVLSDFK

SEQ ID NO: 15 - protein, 18 aa, Artificial Sequence
  Synthetic
SYLREIWDYISEVLSDFR

SEQ ID NO: 16 - protein, 18 aa, Artificial Sequence
  Synthetic
SWLREIWDWICEVLSDFK

SEQ ID NO: 17 - protein, 18 aa, Artificial Sequence
  Synthetic
  MISC_FEATURE (1)..(1): Ac
  MISC_FEATURE (18)..(18): NH2
SWLRDIWDWICEVLSDFK

SEQ ID NO: 18 - protein, 18 aa, Artificial Sequence
  Synthetic
  MISC_FEATURE (1)..(1): Ac
  MISC_FEATURE (18)..(18): NH2
SWLRDIWDWICEVLSDFK

SEQ ID NO: 19 - protein, 19 aa, Artificial Sequence
  Synthetic
  MISC_FEATURE (1)..(1): Ac
  MISC_FEATURE (19)..(19): NH2
GSWLRDIWDWICEVLSDFK

SEQ ID NO: 20 - protein, 20 aa, Artificial Sequence
  Synthetic
  MISC_FEATURE (1)..(1): Ac
  MISC_FEATURE (20)..(20): NH2
SWLRDIWDWICEVLSDFKTW

SEQ ID NO: 21 - protein, 18 aa, Artificial Sequence
  Synthetic
  MISC_FEATURE (1)..(1): Ac
  MISC_FEATURE (18)..(18): NH2
KFDSLVECIWDWIDRLWS

SEQ ID NO: 22 - protein, 18 aa, Artificial Sequence
  Synthetic
  MISC_FEATURE (1)..(1): Ac
  MISC_FEATURE (18)..(18): NH2
KWLCRIWSWISDVLDDFE

SEQ ID NO: 23 - protein, 18 aa, Artificial Sequence
  Synthetic
  MISC_FEATURE (1)..(1): Ac
  MISC_FEATURE (18)..(18): NH2
SIWRDWVDLICEFLSDWK

SEQ ID NO: 24 - protein, 18 aa, Artificial Sequence
  Synthetic
  MISC_FEATURE (1)..(1): Ac
  MISC_FEATURE (18)..(18): NH2
DWLRIIWDWVCSVVSDFK

SEQ ID NO: 25 - protein, 18 aa, Artificial Sequence
  Synthetic
  MISC_FEATURE (1)..(1): Ac
  MISC_FEATURE (18)..(18): NH2
SWLRDIWDWISEVLSDFK

SEQ ID NO: 26 - protein, 18 aa, Artificial Sequence
  Synthetic
  MISC_FEATURE (1)..(1): Ac
  MISC_FEATURE (18)..(18): NH2
SWLRDIWDWISEVLSDFK

SEQ ID NO: 27 - protein, 18 aa, Artificial Sequence
  Synthetic
  MISC_FEATURE (1)..(1): Ac
  MISC_FEATURE (18)..(18): NH2
SWLRDIWDWICEVLSDFR

SEQ ID NO: 28 - protein, 18 aa, Artificial Sequence
  Synthetic
  MISC_FEATURE (1)..(1): Ac
  MISC_FEATURE (18)..(18): NH2
SYLRDIWDYICEVLSDFK

SEQ ID NO: 29 - protein, 18 aa, Artificial Sequence
  Synthetic
  MISC_FEATURE (1)..(1): Ac
  MISC_FEATURE (18)..(18): NH2
SYLRDIWDYISEVLSDFK

SEQ ID NO: 30 - protein, 18 aa, Artificial Sequence
  Synthetic
  MISC_FEATURE (1)..(1): Ac
  MISC_FEATURE (18)..(18): NH2
SYLREIWDYISEVLSDFR

SEQ ID NO: 31 - protein, 18 aa, Artificial Sequence
  Synthetic
  MISC_FEATURE (1)..(1): Ac
  MISC_FEATURE (18)..(18): NH2
SWLREIWDWICEVLSDFK

SEQ ID NO: 32 - protein, 18 aa, Artificial Sequence
  Synthetic
SWLRDIWDWISEVLSDFR

SEQ ID NO: 33 - protein, 18 aa, Artificial Sequence
  Synthetic
SWLRDIWDYISEVLSDFR

SEQ ID NO: 34 - protein, 18 aa, Artificial Sequence
  Synthetic
SWLRDIWDYISEVLSDFR

SEQ ID NO: 35 - protein, 18 aa, Artificial Sequence
  Synthetic
SWLRDIWDYICEVLSDFR

SEQ ID NO: 36 - protein, 18 aa, Artificial Sequence
  Synthetic
  MISC_FEATURE (1)..(1): Ac
  MISC_FEATURE (18)..(18): NH2
SWLRDIWDWISEVLSDFR

SEQ ID NO: 37 - protein, 18 aa, Artificial Sequence
  Synthetic
  MISC_FEATURE (1)..(1): Ac
  MISC_FEATURE (18)..(18): NH2
SWLRDIWDYISEVLSDFR

SEQ ID NO: 38 - protein, 18 aa, Artificial Sequence
  Synthetic
  MISC_FEATURE (1)..(1): Ac
  MISC_FEATURE (18)..(18): NH2
SWLRDIWDYISEVLSDFR

SEQ ID NO: 39 - protein, 18 aa, Artificial Sequence
  Synthetic
  MISC_FEATURE (1)..(1): Ac
  MISC_FEATURE (18)..(18): NH2
SWLRDIWDYICEVLSDFR

SEQ ID NO: 40 - protein, 18 aa, Artificial Sequence
  Synthetic
AWLRDIWDWICEVLSDFK

SEQ ID NO: 41 - protein, 18 aa, Artificial Sequence
  Synthetic
SALRDIWDWICEVLSDFK

SEQ ID NO: 42 - protein, 18 aa, Artificial Sequence
  Synthetic
SWARDIWDWICEVLSDFK

SEQ ID NO: 43 - protein, 18 aa, Artificial Sequence
  Synthetic
SWLADIWDWICEVLSDFK

SEQ ID NO: 44 - protein, 18 aa, Artificial Sequence
  Synthetic
SWLRAIWDWICEVLSDFK

SEQ ID NO: 45 - protein, 18 aa, Artificial Sequence
  Synthetic
SWLRDAWDWICEVLSDFK

SEQ ID NO: 46 - protein, 18 aa, Artificial Sequence
  Synthetic
SWLRDIADWICEVLSDFK

SEQ ID NO: 47 - protein, 18 aa, Artificial Sequence
  Synthetic
SWLRDIWAWICEVLSDFK

SEQ ID NO: 48 - protein, 18 aa, Artificial Sequence
  Synthetic
SWLRDIWDAICEVLSDFK

SEQ ID NO: 49 - protein, 18 aa, Artificial Sequence
  Synthetic
SWLRDIWDWACEVLSDFK

SEQ ID NO: 50 - protein, 18 aa, Artificial Sequence
  Synthetic
SWLRDIWDWIAEVLSDFK

SEQ ID NO: 51 - protein, 18 aa, Artificial Sequence
  Synthetic
SWLRDIWDWICAVLSDFK

SEQ ID NO: 52 - protein, 18 aa, Artificial Sequence
  Synthetic
SWLRDIWDWICEALSDFK

SEQ ID NO: 53 - protein, 18 aa, Artificial Sequence
  Synthetic
SWLRDIWDWICEVASDFK

SEQ ID NO: 54 - protein, 18 aa, Artificial Sequence
  Synthetic
SWLRDIWDWICEVLADFK

SEQ ID NO: 55 - protein, 18 aa, Artificial Sequence
  Synthetic
SWLRDIWDWICEVLSAFK

SEQ ID NO: 56 - protein, 18 aa, Artificial Sequence
  Synthetic
SWLRDIWDWICEVLSDAK

SEQ ID NO: 57 - protein, 18 aa, Artificial Sequence
  Synthetic
SWLRDIWDWICEVLSDFA

SEQ ID NO: 58 - protein, 19 aa, Artificial Sequence
  Synthetic
  MISC_FEATURE (1)..(1): Ac
SWLRDIWDWICEVLSDFKK

SEQ ID NO: 59 - protein, 19 aa, Artificial Sequence
  Synthetic
  MISC_FEATURE (19)..(19): NH2
KSWLRDIWDWICEVLSDFK

SEQ ID NO: 60 - protein, 19 aa, Artificial Sequence
  Synthetic
  MISC_FEATURE (1)..(1): Ac
  MISC_FEATURE (1)..(1): palmitoyl
  MISC_FEATURE (19)..(19): NH2
KSWLRDIWDWICEVLSDFK

SEQ ID NO: 61 - protein, 19 aa, Artificial Sequence
  Synthetic
  MISC_FEATURE (1)..(1): Ac
  MISC_FEATURE (19)..(19): Palmitoyl
  MISC_FEATURE (19)..(19): NH2
SWLRDIWDWICEVLSDFKK

SEQ ID NO: 62 - protein, 18 aa, Artificial Sequence
  Synthetic
  MISC_FEATURE (1)..(1): Ac
  MISC_FEATURE (18)..(18): amino-PEG12-propionyl-NH2
SWLRDIWDWICEVLSDFK

SEQ ID NO: 63 - protein, 18 aa, Artificial Sequence
  Synthetic
  MISC_FEATURE (1)..(1): Ac-amino-PEG12-propionyl
  MISC_FEATURE (18)..(18): NH2
SWLRDIWDWICEVLSDFK

SEQ ID NO: 64 - protein, 19 aa, Artificial Sequence
  Synthetic
  MISC_FEATURE (1)..(1): Ac
  MISC_FEATURE (19)..(19): (succinimido-propionylaminoethyl-mPEG2K)-NH2
SWLRDIWDWICEVLSDFKC

SEQ ID NO: 65 - protein, 19 aa, Artificial Sequence
  Synthetic
  MISC_FEATURE (1)..(1): Ac
  MISC_FEATURE (1)..(1): (succinimido-propionylaminoethyl-mPEG2K)
  MISC_FEATURE (19)..(19): NH2
CSWLRDIWDWICEVLSDFK

SEQ ID NO: 66 - protein, 20 aa, Artificial Sequence
  Synthetic
  MISC_FEATURE (2)..(2): oleoyl
  MISC_FEATURE (20)..(20): NH2
HKSWLRDIWDWICEVLSDFK

SEQ ID NO: 67 - protein, 19 aa, Artificial Sequence
  Synthetic
  MISC_FEATURE (1)..(1): Ac
  MISC_FEATURE (19)..(19): (oleoyl)-NH2
SWLRDIWDWICEVLSDFKK

SEQ ID NO: 68 - protein, 20 aa, Artificial Sequence
  Synthetic
  MISC_FEATURE (2)..(2): cholesteryloxycarbonylmethyl
  MISC_FEATURE (20)..(20): NH2
HCSWLRDIWDWICEVLSDFK

SEQ ID NO: 69 - protein, 19 aa, Artificial Sequence
  Synthetic
  MISC_FEATURE (1)..(1): Ac
  MISC_FEATURE (19)..(19): (cholesteryloxycarbonylmethyl)-NH2
SWLRDIWDWICEVLSDFKC

SEQ ID NO: 70 - protein, 19 aa, Artificial Sequence
  Synthetic
  MISC_FEATURE (1)..(1): Ac
  MISC_FEATURE (1)..(1): (succinyl-D-a-tocopherol)
  MISC_FEATURE (19)..(19): (succinyl-D-a-tocopherol)
KSWLRDIWDWICEVLSDFK

SEQ ID NO: 71 - protein, 19 aa, Artificial Sequence
  Synthetic
  MISC_FEATURE (1)..(1): Ac
  MISC_FEATURE (19)..(19): (succinyl-D-a-tocopherol)-NH2
SWLRDIWDWICEVLSDFKK

SEQ ID NO: 72 - protein, 18 aa, Artificial Sequence
  Synthetic
  MISC_FEATURE (1)..(1): Xaa is S, K, D, A, N, M, T, or V
  MISC_FEATURE (2)..(2): Xaa is W, I, Y, F, P, L, V, H, M, A, T, or S
  MISC_FEATURE (3)..(3): Xaa is L, W, I, M, V, F, M, A, or T
  MISC_FEATURE (4)..(4): Xaa is R, C, K, Q, H, P, or C
  MISC_FEATURE (5)..(5): Xaa is D, R, I, E, N, or Y
  MISC_FEATURE (6)..(6): Xaa is I, W, V, L, or F
  MISC_FEATURE (7)..(7): Xaa is W, V, Y, F, P, L, V, H, or D
  MISC_FEATURE (8)..(8): Xaa is W, L, Y, F, P, L, V, or H
  MISC_FEATURE (9)..(9): Xaa is W, L, Y, F, P, L, V, or H
  MISC_FEATURE (10)..(10): Xaa is I, V, L, or F
  MISC_FEATURE (11)..(11): Xaa is C, S, A, P, M, H, or T
  MISC_FEATURE (12)..(12): Xaa is E, D, S, Q, Y, or T
  MISC_FEATURE (13)..(13): Xaa is V, F, I, L, A, F, or M
  MISC_FEATURE (14)..(14): Xaa is L, V, I, M, or F
  MISC_FEATURE (15)..(15): Xaa is S, D, A, N, T, Y, or P
  MISC_FEATURE (16)..(16): Xaa is D, E, N, Y, or S
  MISC_FEATURE (17)..(17): Xaa is F, W, Y, L, P, or H
  MISC_FEATURE (18)..(18): Xaa is K, E, R, H, R, P, or Q
XXXXXXXXXXXXXXXXXX

SEQ ID NO: 73 - protein, 6 aa, Artificial Sequence
  Synthetic
GSGSGC

SEQ ID NO: 74 - DNA, 113 bp, Artificial Sequence
  Synthetic
acaggtacgttaatagttaatagcgtacttctttttcttgctttcgtggtattcttgctagttacactagccatccttactgcgcttcgattgtgtgcgtactgctgcaatat

SEQ ID NO: 75 - DNA, 91 bp, Artificial Sequence
  Synthetic
aatcagcacctttaattgaattgtgcgtggatgaggctggttctaaatcacccattcagtacatcgatatcggtaattatacagtttcctg

SEQ ID NO: 76 - DNA, 19 bp, Artificial Sequence
  Synthetic
agatttggacctgcgagcg

SEQ ID NO: 77 - DNA, 20 bp, Artificial Sequence
  Synthetic
gagcggctgtctccacaagt

SEQ ID NO: 78 - protein, 38 aa, Artificial Sequence
  Synthetic
  MISC_FEATURE (1)..(1): Ac
  MISC_FEATURE (19)..(19): Xaa is Lys, Ser, Cys
  MISC_FEATURE (20)..(20): Xaa is Lys, Ser, Cys
  MISC_FEATURE (38)..(38): NH2
SWLRDIWDWICEVLSDFKXXSWLRDIWDWICEVLSDFK